(19) United States
(12) Patent Application Publication
(10) Pub. No.: US 2022/0028486 A1
(43) Pub. Date: Jan. 27, 2022

(54) SYSTEMS AND METHODS FOR DETERMINING A PHYSIOLOGICAL PROFILE USING GENETIC INFORMATION

(71) Applicant: The DNA Company Inc., Mississauga, CA (US)

(72) Inventors: Mansoor Mohammed, Oakville (CA); David Liepert, Calgary (CA); Harris Khan, Mississauga (CA); Kashif Khan, Mississauga (CA); Kashif Siddiqui, Mississauga (CA)

(73) Assignee: The DNA Company Inc., Mississauga, ON (CA)

(21) Appl. No.: 17/382,472

(22) Filed: Jul. 22, 2021

Related U.S. Application Data
(60) Provisional application No. 63/167,830, filed on Mar. 30, 2021.
(60) Provisional application No. 63/055,518, filed on Jul. 23, 2020.

(51) Int. Cl. G16B 20/20; G16H 10/60; G16H 50/30

(52) U.S. Cl. CPC G16B 20/20; G16H 50/30; G16H 10/60

(57) ABSTRACT

A system for providing intervention information to a subject may be configured to perform a method including: receiving, via the processor, genetic information of the subject, the genetic information including a plurality of sets, and each set comprising one or more genes; assigning, via the processor, a value to each respective set; assigning, via the processor, a respective classification to the subject in one or more categories, wherein: each category corresponds to a respective set; and the assignment of the respective classification is based on the respective values assigned to the respective sets; obtaining, via the processor, health profile information and wellness intervention information associated with the assigned classifications of the one or more categories; and outputting, via the processor, the health profile information and the wellness intervention information to a user. The assignment of the respective classification may be predictive of the health state of the subject, and a method may include: generating a health profile of the subject based on the predicted health state from the assigned classifications for the one or more categories; and outputting the health profile of the subject.

### RELATED APPLICATIONS

This application claims the benefit of priority to U.S. Provisional Application No. 63/055,518, entitled “SYSTEMS AND METHODS FOR DETERMINING A PHYSIOLOGICAL PROFILE USING GENETIC INFORMATION,” filed Jul. 23, 2020, and to U.S. Provisional Application No. 63/167,830, entitled “SYSTEMS AND METHODS FOR DETERMINING A PHYSIOLOGICAL PROFILE USING GENETIC INFORMATION,” filed Mar. 30, 2021, the disclosures of which are incorporated herein in their entirety.

### TECHNICAL FIELD

Various embodiments of the present disclosure relate generally to determining physiological information and/or a medical or other intervention for a subject, and, in some embodiments, relate particularly to methods and systems for using a genotype-based model to identify a profile classification for the subject and/or identify a medical intervention for the subject based on the subject's profile classification.

### BACKGROUND

A person's genes, collectively known as the person's genome, encode a blueprint for a multitude of physiological traits that may affect not only the person's development, health, and wellbeing, but also the person's interaction with their environment. While some genes have been associated with particular physiological traits, risk factors, or disorders, many genes may be interrelated in ways that are not well understood. For example, an association between a particular trait and an expression of one or more genes (i.e., a genotype) may be based on a statistical relationship, e.g., a patient study indicating that subjects with a particular trait are statistically correlated with having a particular genotype. However, such a study does not address complex gene interactions that may be present, or how the genotype may be related to various subject health factors. Without a deeper understanding of such factors, a person's genome may be of limited predictive or interventional value.

The present disclosure is directed to addressing one or more of these above-referenced challenges or other challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

According to certain aspects of the disclosure, methods and systems are disclosed for identifying one or more profile classifications for a subject and/or identifying one or more medical or other interventions for the subject based on the subject's profile classification(s).

In one aspect, an exemplary computer-implemented method for providing wellness intervention information to a subject may include: receiving, via a processor of a user device associated with a user, genetic information of the subject, the genetic information including a plurality of sets, and each set comprising one or more genes; assigning, via the processor, a value to each respective set; assigning, via the processor, a respective classification to the subject in one or more categories, wherein: each category corresponds to a respective set; and the assignment of the respective classification is based on the respective values assigned to the respective sets; obtaining, via the processor, health profile information and wellness intervention information associated with the assigned classifications of the one or more categories; and outputting, via the processor, the health profile information and the wellness intervention information to the user via the user device.

In another aspect, an exemplary system for providing intervention information to a subject may include: a processor; and a memory operatively connected to the processor and storing instructions that, when executed by the processor, cause the processor to perform acts including: receiving, via the processor, genetic information of the subject, the genetic information including a plurality of sets, and each set comprising one or more genes; assigning, via the processor, a value to each respective set; assigning, via the processor, a respective classification to the subject in one or more categories, wherein: each category corresponds to a respective set; and the assignment of the respective classification is based on the respective values assigned to the respective sets; obtaining, via the processor, health profile information and wellness intervention information associated with the assigned classifications of the one or more categories; and outputting, via the processor, the health profile information and the wellness intervention information to a user.

In a further aspect, an exemplary method of predicting a health state of a subject may include: receiving genetic information of the subject, the genetic information including a plurality of sets, and each set comprising one or more genes; assigning a value to each respective set; assigning a respective classification to the subject in one or more categories, wherein: each category corresponds to a respective set; the assignment of the respective classification is based on the respective values assigned to the respective sets; and the assignment of the respective classification is predictive of the health state of the subject; generating a health profile of the subject based on the predicted health state from the assigned classifications for the one or more categories; and providing the health profile of the subject to the subject.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary embodiment of a computing environment that may be used to implement one or more aspects of the present disclosure.

FIG. 2 depicts an exemplary embodiment of a process for developing a genetic profile of a subject.

FIG. 3 depicts another exemplary embodiment of a process for developing a genetic profile of a subject.

FIG. 4 depicts a further exemplary embodiment of a process for developing a genetic profile of a subject.

FIG. 5 depicts an example of a computing device, according to aspects of the present disclosure.

### DETAILED DESCRIPTION OF EMBODIMENTS

The terminology used in this disclosure is to be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. The following description is exemplary and explanatory only and is not restrictive of the features described with regard to any particular embodiment or example.

In this disclosure, the term “based on” means “based at least in part on.” The singular forms “a,” “an,” and “the” include plural referents unless the context dictates otherwise. The term “exemplary” is used in the sense of “example” rather than “ideal.” The term “or” is meant to be inclusive and means either, any, several, or all of the listed items. The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as, “substantially,” “approximately,” “about,” and “generally,” are used to indicate a possible variation of ±10% of a stated or understood value.

In this disclosure, the term “computer system” generally encompasses any device or combination of devices, each device having at least one processor that executes instructions from a memory medium. Additionally, a computer system may be included as a part of another computer system. An “electronic application” generally encompasses a program, website, service, interface, or the like that may be accessed by a user via an electronic device such as a computer system like a desktop computer, mobile device, or the like in order to provide goods, services, information, or the like to the user.

As used herein, the term “provider” may indicate, and may be used interchangeably with, a doctor, hospital, medical insurance provider, health institution such as a governmental heath institution or regulatory entity, a source or vendor of medical items, services, or information, or the like. The term “user” may indicate, and may be used interchangeably with, a customer, patient, subject, person with a medical issue and/or a person seeking information, treatment, intervention, or the like associated with a medical issue. The term “medical intervention” generally encompasses medication, a supplement, a therapy, an activity, a procedure, a surgery, a lifestyle change, or the like, or combinations thereof, associated with an effect on a subject's physical and/or mental health or wellbeing. A medical intervention may include an action for the subject before and/or after the subject may have been affected by a malady. As used herein, a “malady” generally encompasses physical, physiological, and/or mental conditions, ailments, disorders, syndromes, or disabilities known now or in the future. A malady may include a genetic disorder, a risk factor, a susceptibility, a condition to which the subject may be more or less at risk due to a genetic predisposition, a condition influenced or caused at least in part by one or more factors external to the subject's body, or the like.

According to certain aspects of the disclosure, methods, systems, and non-transitory computer-readable media are disclosed for developing a genetic profile of a subject, e.g., by determining one or more genetic classifications for the subject, and/or recommending or directing one or more medical interventions for the subject based on the developed genetic profile. Each of the examples disclosed herein may include one or more of the features described in connection with any of the other disclosed examples.

In general, the present disclosure provides methods and systems for determining physiological information and/or a medical intervention for a subject. In some embodiments, a genotype-based model may be employed to develop a profile for the subject by assigning one or more classifications for the subject in one or more categories based on the subject's genetic information. As used herein, a “category” may be associated with one or more physical, mental, physiological aspects, risk factors, susceptibilities, illnesses, or the like, whereby a “classification” corresponds to a particular variation or expression of the category for the subject.

A particular classification in a category may be indicated by one or more values of one or more genetic indicators associated with the category. As used herein, a “genetic indicator” is one or more genes that, as a whole, act as a building block for a classification or otherwise factor into that classification. Thus, each category may be associated with a set of portions of genetic information of a subject, whereby each portion in a set corresponds to a particular genetic indicator.

In some embodiments, an indicator may be associated with one or more genes and/or one or more other indicators. In some embodiments, a category may act as an indicator for another category. Additionally, a genetic indicator has a value that is based on a particular expression of the one or more associated genes and/or values of other associated indicators. For example, a first indicator may have a value of “TRUE” or “FALSE” depending on the genotypes of three associated genes, a second indicator may have a value of “FAST” or “SLOW” depending on the genotypes of two associated genes, and a third indicator may have a value of “INCREASED RISK” or “DECREASED RISK” based on the values of the first and second indicators.

Values for genetic indicators may be binary (e.g., yes or no, active or inactive, present or not present, etc.), qualitative (e.g., fast, medium, or slow, etc.), quantitative (e.g., weights, scores, etc.), or combinations thereof. In various embodiments, the values for different indicators associated with a category may be of the same form, e.g., each a selection of “FAST” or “SLOW”, or may be of different forms, e.g., one indicator has a value selected from “FAST” or “SLOW” while another has a value selected from “Optimal”, “Average”, or “Suboptimal”, etc. In some embodiments, a value for an indicator may function as a selection in a decision tree. For example, a first value for an indicator may reduce a set of classifications for a category to a first subset of classification, and a second value of the indicator may reduce the set of classification to a second subset. In some embodiments, the first and second subsets may overlap. In some embodiments, there may be no overlap between subsets. Any suitable schema for values of indicators may be used.

Since a classification is associated with a trait and/or a risk factor for a trait, assigning a classification of a subject for a category may include and/or may be associated with predicting a health state of the subject. For example, if a subject is classified as high risk for breast cancer, e.g., based on value(s) of indicator(s) associated with a category for breast cancer risk, the assignment of that classification may act as a prediction of the health state of the subject (e.g., at risk of breast cancer), based on the value(s) of the indicator(s), and thus based on the genetic information of the subject.

Genetic testing has been used to diagnose various illnesses and to understand more about the traits of an individual. However, conventional genetic testing techniques generally rely on statistical relationships between genes and traits or illnesses, and generally do not account for complex gene interactions. Further, conventional techniques may be of limited value for providing information associated with traits of the subject, or for recommending or enacting a medical or other intervention, e.g., a wellness intervention, for the subject. Accordingly, a need exists to improve the understanding of a causal relation between genes and traits. A need also exists to improve the selection of medical interventions for a subject based on their individual genotype.

In an exemplary use case, a subject and/or a provider may desire to understand how a subject's hormone balance and resulting traits may be impacted by their genotype, and/or recommend or enact one or more wellness interventions for the subject based on their genotype. A delicate balance of reproductive hormones may play a significant role in a person's overall health. Hormones are chemical messengers produced and released into the bloodstream by endocrine glands throughout the body. Once in the bloodstream, hormones are delivered to cells, where they exert considerable effects on function, growth, and development. Specifically, reproductive or sex hormones (SH) are hormones that play an essential role in sexual development and reproduction. The majority of SH in men and women are produced by the respective reproductive organs—testes in males and ovaries in females. SH are not exclusively produced by the reproductive organs, however, as small amounts are also produced by adrenal glands (small glands sitting on top of the kidneys). Production and balance of SH is controlled by a small gland at the base of the brain called the pituitary gland, which in turn is controlled by a region of the brain called the hypothalamus.

There are three primary classes of sex hormones: progesterones, androgens (like testosterone), and estrogens. In general, androgens are responsible for the stimulation and development of male characteristics, and estrogens for female characteristics. However, the delicate balance of these hormones may be significantly impacted by a person's individual genotype in a myriad of ways, and may result in a variety of traits.

To better understand how the subject's hormone balance and resulting traits may be impacted by their genotype, and/or to recommend or enact one or more medical or other interventions for the subject based on their genotype, the subject and/or the provider may employ a genotype-based model to develop a genetic profile for the subject. In some examples, the subject and/or the provider (i.e., a user) may access an assessment tool, e.g. via an electronic application, a website page, or the like. The assessment tool may prompt the user to answer one or more preliminary questions, such as demographic questions, location questions, lifestyle questions, current health status questions, or the like. The assessment tool may prompt the user for account information, privacy and/or medical information consent, or other authentication related information, as discussed in further detail below. The assessment tool may prompt the user to provide genetic information for the subject, e.g., via a file upload, or may retrieve such genetic information from another source such as a memory or a remote system. The genotype-based model may use the provided genetic information and/or provided answers to the preliminary questions to develop the genetic profile for the subject. Developing the profile may include, for example, applying the subject's genotype to the genotype-based model to assign one or more classifications to the subject.

In some examples, the profile may include a classification for the subject in the category of reproductive hormone balance, whereby possible classifications may include androgen dominant, estrogen dominance, estrogen-balanced (e.g., balanced or no dominance, favoring estrogen), androgen-balanced (e.g., balanced or no dominance, favoring androgen), or co-dominant. Assigning a classification to the subject for a particular category may include, for example, determining one or more values of one or more indicators associated with the category, and determining a classification based on the one or more values, as discussed in more detail below. In some examples, the assignment of a classification for the subject in a category includes a prediction of health state of the subject.

Each category may be associated with one or more traits such as, for example, body type, hormone production levels or balance, etc. Each classification may also be associated with an expression of such a trait and/or one or more risk factors for occurrence of such a trait. For example, for a male subject, a classification may be associated with an increased, normal, or decreased risk of low testosterone, male-pattern baldness, benign prostate hyperplasia, lean muscle, low libido, consequences resulting from hormone replacement, etc. In another example, for a female subject, a classification may be associated with a presence or risk for one or more illnesses or conditions such as, for example, premature ovarian failure, irregular ovulation, risk of estrogen toxicity, endometriosis/fibroids, polycystic ovarian syndrome, vascular or neurological inflammation, etc. Each classification may also be associated with one or more wellness interventions.

In other exemplary use cases, the profile may include classifications in other categories. For instance, a genotype-based model may be employed to gain insight as to how a subject's body clears out toxins, responds to environmental effects like pollutants, allergens, etc., how their body is susceptible to and/or responds to inflammation, how their body's metabolism operates, and/or risk factors for various illnesses or conditions such as chemical sensitivity, fibromyalgia, neuropathy, autoimmune conditions, chronic fatigue, susceptibility to disease infection, or mental disorders like anxiety, stress, depression, addiction, irritability, neuroticism, etc.

The assessment tool may display the genetic profile to the subject and/or the medical provider. In some examples, the assessment tool may display information associated with one or more genes, indicators, classifications, etc., for the subject, as well as associated traits, risk factors, or interventions. In some examples, the assessment tool may display information associated with health state predictions for the subject, and or an association between a predicted health state and the subject's genetic information, e.g., via one or more indicators, classifications, or categories.

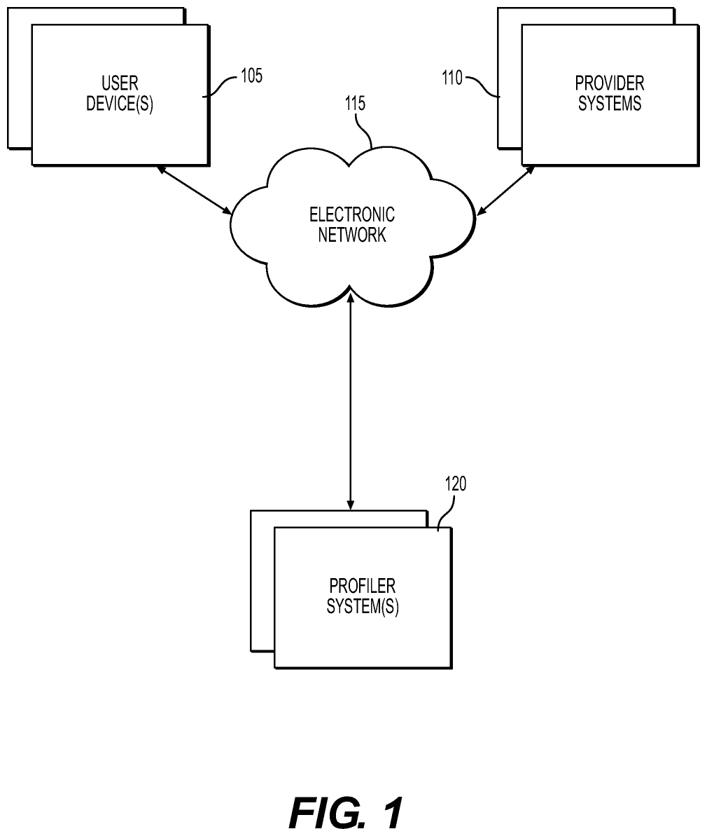

FIG. 1 depicts an exemplary client-server environment that may be utilized with techniques presented herein. One or more user device(s) 105 and/or one or more provider system(s) 110 may communicate across an electronic network 115. The systems of FIG. 1 may communicate in any arrangement. The user device 105 may be associated with a user, e.g., a subject or provider seeking information, treatment, intervention, or the like associated with the subject's genetic information. The user device 105 may be operated by a subject, by a medical provider or other intermediary associated with the subject, or combinations thereof.

As will be discussed herein, one or more profiler system(s) 120 may communicate with each other and/or with the user device 105 and/or the provider system 110 over the electronic network 115 in providing an assessment tool for determining a genetic profile for the subject. In some embodiments, the assessment tool may employ a genotype-based model in order to, for example, develop or update a genetic profile. As used herein, a “model” may include data (e.g., illness data, subject medical data, trait data, genotype data, demographic data, classification data, indicators data, or historical user data) and/or instruction(s) for generating, retrieving, and/or analyzing such data.

In various embodiments, the electronic network 115 may be a wide area network (“WAN”), a local area network (“LAN”), personal area network (“PAN”), or the like. In some embodiments, electronic network 115 includes the Internet, and information and data provided between various systems occurs online. “Online” may mean connecting to or accessing source data or information from a location remote from other devices or networks coupled to the internet. Alternatively, “online” may refer to connecting or accessing an electronic network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”).

While FIG. 1 depicts the various systems as physically separate and communicating across network 115, in various embodiments features of certain systems, such as the profiler system 120, may be incorporated partially or completely into any of the other systems of FIG. 1. For example, the profiler system 120 may be incorporated by the provider system 110 and/or the user device 105. In another example, the user device 105 may be incorporated into the provider system 110, or vice versa. In some embodiments, some or all of the functionality of the genotype-based model may be incorporated into the user device 105. In some embodiments, some or all of the functionality of the profiler system 120 may be incorporated into an electronic application or website accessible by the user device 105.

Several processes associated with providing a genetic profile of a subject are discussed below. It should be understood that such processes are exemplary only, and that other processes according to this disclosure may omit one or more of the steps discussed below, may include additional steps, and may arrange steps in any suitable order.

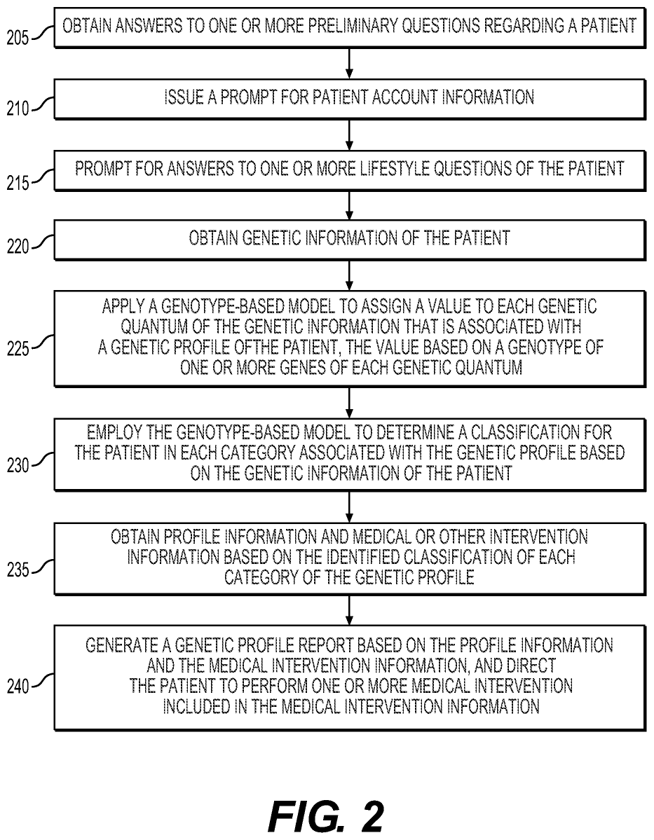

FIG. 2 illustrates an exemplary process for providing a genetic profile of a subject to a user. A user desiring a genetic profile of the subject may access an assessment tool provided by the profiler system 120, e.g., via a user device 105 or a provider system 110, or the like. The assessment tool may employ an electronic application, a software program, a website page, or the like. It should be understood that, in various embodiments, actions described as performed by the assessment tool may be performed by, for example, the profiler system 120, the user device 105, the provider system 110, another system, or combinations thereof.

At step 205, the assessment tool may obtain answers to one or more preliminary questions regarding the subject. In various embodiments, the preliminary questions may include, for example, non-personally identifying questions such as, for example, demographic questions, location questions, or the like. In some embodiments, in conjunction with obtaining the answers to the preliminary questions, the assessment tool may provide information related to answers received for the preliminary questions and/or other information associated with the genetic profile.

In some embodiments, the assessment tool may prompt the user to answer the one or more preliminary questions. In some embodiments, instead of or in addition to prompting for answers for the preliminary questions, the assessment tool may retrieve information associated with the preliminary questions from one or more sources. For example, the assessment tool may retrieve information from the user device such as location information, information from a provider system 110 such as profile, medical, or genetic information associated with the subject. In some embodiments, such prompting and/or retrieval is performed in response to receiving user approval, a user login request, an authentication of the user, or the like.

For example, in some embodiments, at step 210, the assessment tool may issue a prompt for subject account information. Such a prompt may include one or more of a request for existing account information, a request for consent to use personally identifying information and/or medical information associated with the subject, a prompt to register a new account for the subject, a prompt for authenticating the user, information related to information privacy and data use, or the like. In some embodiments, in response to information and/or consent received at step 210, the assessment tool may retrieve subject profile information and/or subject medical information from at least one source such as, for example, the provider system 110, a memory of the user device 105, a memory of the assessment system 120, a social information source like a social media profile of the subject, or the like. Subject profile information may include descriptive information associated with the subject such as age, gender, location, marital status, family and/or social connections, employment, hobbies, activities, travel history, etc. Subject medical information may include medical history data, medical test or diagnostic data, physiological or genetic data, or the like.

At step 215, the assessment tool may prompt for answers to lifestyle questions of the subject. A lifestyle question is a question having an answer that may be probative about an environment or condition that the subject may be exposed to. In some embodiments, the assessment tool may provide general information and/or recommendations based on positive responses to one or more of the lifestyle questions.

At step 220, the assessment tool may obtain genetic information of the subject. In various embodiments, the assessment tool may obtain the genetic information via data uploaded to the profiler system 120, and/or via retrieval from memory of the profiler system 120, the provider system 110, the user device 105, or another system, etc. In some embodiments, portions of the genetic information may be obtained from different sources. In some embodiments, at least a portion of genetic information is obtained via obtaining answers to phenotype questions for the subject, and applying one or more relational models to such answers to identify genotypes of the subject. Further details of such features are discussed in commonly owned U.S. Provisional Patent No. 63/021,237 filed on May 21, 2020, entitled “Systems and Methods for Determining Symptom Severity Risk and Intervention Recommendation,” the entirety of which is incorporated by reference herein.

The genetic information may include, for example, a genotype for one or more genes of the subject. In some embodiments, one or more categories of the genetic profile to be developed is associated with one or more genetic indicators that are associated with one or more genes, and the genetic information includes genotypes for such one or more genes. In some embodiments, the assessment tool extracts genotypes for the one or more genes from the genetic information. In an illustrative example, a category for the genetic profile, e.g., hormone dominance, may be associated with a plurality of genetic indicators, one of which is associated with the CYP17A1 gene, and the assessment tool may obtain a genotype for the subject of GG for the CYP17A1 gene.

In some embodiments, at step 225, the assessment tool may apply the genotype-based model to assign a value to each genetic indicator associated with the genetic profile based on the genotypes of the one or more genes. Each indicator may have a value that depends on the genotypes of the one or more genes associated with that indicator. In an illustrative example, the indicator discussed above for the CYP17A1 gene may have a value of “FAST” for genotypes of GG or AG, or a value of “SLOW” for the genotype of AA.

At step 230, the assessment tool may employ the genotype-based model to determine a classification for the subject in each category associated with the genetic profile based on the genetic information for the subject. In some embodiments, the classification for a category is determined based on the values of the one or more genetic indicators associated with the category. In an illustrative example, a genetic indicator associated the category of risk of estrogen toxicity in a female subject may be associated with three genes, CYP1A1, CYP1B1, and CYP3A4, and the value of that genetic indicator may include the genotypes of the three associated genes. The assessment tool may determine the classification for the category of risk of estrogen toxicity based on the value of the indicator, e.g. the genotypes of the three associated genes. For example, if CYP1A1 has a genotype of AG, GG, or AA, CYP1B has a genotype of CC, and CYP3A4 has a genotype of AA, the classification for the category is “DECREASED”, and if the genotypes of the three genes do not match the foregoing, the classification for the category is “INCREASED.”

In some embodiments, determining the classification includes, acts as, and/or is associated with making a health state prediction of the subject. For example, a subject may be predicted to exhibit and/or be at risk for the trait(s) and/or conditions associated with a classification.

In some embodiments, determining the classification via the genotype-based model may include applying one or more exception rules. In some embodiments, an exception rule may be applied to a category based on a value of an indicator not otherwise associated with the category. In other words, there may be an exception rule that overrides the value of an indicator and/or a classification associated with the value. For example, continuing from the example above, there may be a further indicator associated with CYP1B1, COMT and an indicator of genes associated with Methylation/Detoxification (discussed in more detail below). If CYP1B1 has a genotype of AG or GG, COMT has a genotype of GG, and the Methylation/Detoxification indicator has a value of “OPTIMAL,” then the classification of the category of risk of estrogen toxicity in a female subject, which would otherwise be “INCREASED” due to the genotype other than CC for the CYP1B1 gene, is overridden to be “DECREASED.”

At step 235, the assessment tool may obtain profile information and medical or other intervention information, e.g., a wellness intervention based on the identified classification of each category of the genetic profile. Profile information may include, for example, a description of traits or characteristics that may be associated with a classification, illnesses or conditions that may be present or that may be associated with an increased risk for the classification, etc. Intervention information may include, for example, one or more medical interventions or other lifestyle, environmental, or other interventions configured to improve the health and well-being of a subject exhibiting the classification and/or to mitigate a risk or illness associated with the classification.

At step 240, the assessment tool may generate a genetic profile report based on the profile information and the medical intervention information and direct the subject, e.g., via transmitting the genetic profile report to the user device 105, to perform the one or more medical or other interventions included in the intervention information. In some embodiments, the genetic profile report may be further based on one or more of the answers to the preliminary questions, lifestyle questions, genetic information, the categories associated with the genetic profile, the determined classifications, the indicators associated with the classifications, etc. In some embodiments, the report may include one or more of a physical document, an electronic document, etc. In some embodiments, the report may include one or more interactive elements, e.g., an interaction with or presentation by a provider or the like, an electronic application, etc. In some embodiments, the report may be integrated into, include, or be associated with a health profile and/or health profile report of the subject.

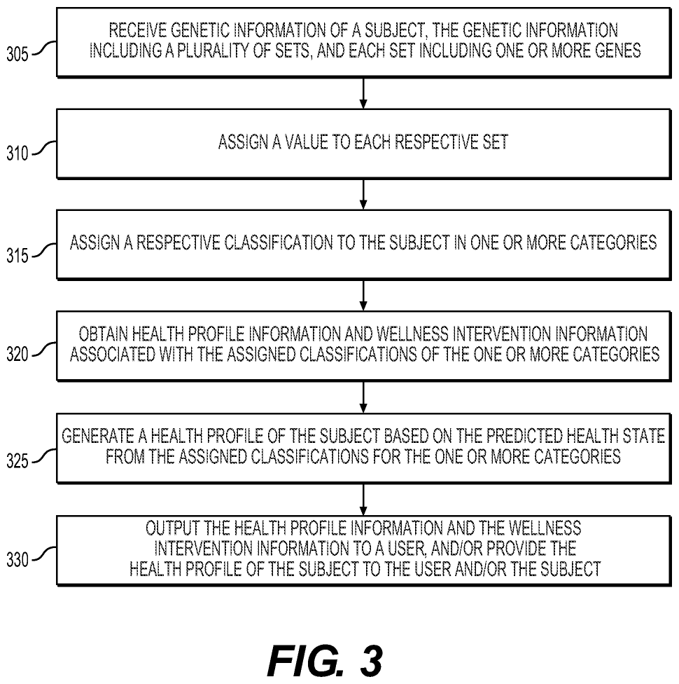

FIG. 3 depicts a flow diagram of another exemplary embodiment of a method for developing a genetic profile of a subject. At step 305, the patient-type recommendation system 130 may receive genetic information of a subject, the genetic information including a plurality of sets, and each set including one or more genes. As used herein, a “set” may be understood to be a group of indicators, or the like.

At step 310, the profiler system 120 may assign a value to each respective set. The assigned values may be based on the genotype of the one or more genes included in each set, e.g., in a manner similar to the procedures discussed elsewhere.

At step 315, the profiler system 120 may assign a respective classification to the subject in one or more categories. Each category may correspond to at least one set. The assignment of the respective classification for each category may be based on the respective values assigned to the at least one corresponding set.

At step 320, the profiler system 120 may obtain health profile information and wellness intervention information associated with the assigned classifications of the one or more categories.

In some embodiments, the assignment of the respective classification to the respective category is predictive of the health state of the subject. At step 325, the profiler system 120 may generate a health profile of the subject based on the predicted health state from the assigned classifications for the one or more categories. In some embodiments, the health profile may include the obtained health profile information and wellness intervention information.

At step 330, the profiler system 120 may output the health profile information and the wellness intervention information to the user via the user device, and/or provide the health profile of the subject to the user and/or the subject.

Various embodiments of the methods above may be applied to develop genetic profiles with a variety of categories. Several examples are provided below, although it should be understood that embodiments of the methods above may be applied to any suitable category or combination of categories known now or in the future.

### Example 1: Female Subject Hormone Report

A Female Subject Hormone Report may provide a personalized summary of genomics that influence reproductive pathways of a female subject, and consequentially the predisposition with which the subject's body maintains the delicate balance of hormones. Such balance may have an impact on (i) the subject's body type, (ii) how likely the subject is to add muscle, have cellulite, or store fat, or (iii) other female health concerns such as endometriosis, polycystic ovarian syndrome, estrogen toxicity, risk associated with hormone replacement (e.g., birth control, etc.), timing and severity of menopausal symptoms, cystic acne, or the like. The Female Subject Hormone Report may also include a recommendation or direction to enact one or more interventions such as, for example, changes to diet, environment, lifestyle, supplements, therapy, medical procedures, etc. For example, introduction of hormones from external sources may exaggerate or influence a patent's hormone balance, and the Report may provide interventions to prevent or mitigate at least a portion of such effects.

To develop a Female Subject Hormone Report, an assessment tool, such as the assessment tool described above with regard to FIG. 2, may obtain genetic information for a female subject. The Report may be associated with one or more of the following categories: Hormone Dominance; Risk of Estrogen Toxicity; Methylation Detoxification; and Glutathionization Detoxification. Tables 1-9 depict an example of various relationships that may be included in a genotype-based model used by the assessment tool. Table 1 below lists an exemplary embodiment of the indicators associated with each category.

TABLE 1
Indicators Associated with Profile Categories

| Category | Indicator Type | Indicators |
|---|---|---|
| Hormone | Gene | CYP17A1 |
| Dominance | Gene | CYP19A1 |
| | Gene | SRD5A2 |
| | Gene | UGT2B17 |
| | Gene | COMT |
| Risk of Estrogen | Gene | CYP1A1 |
| Toxicity | Gene | CYP1 B1 |
| | Gene | CYP3A4 |
| Methylation | Gene | MTHFR |
| Detoxification | Gene | SHMT1 |
| | Gene | MTRR |
| | Gene | MTR |
| | Gene | MTHFR-2 |
| Glutathionization | Gene | GSTT1 |
| Detoxification | Gene | GSTM1 |
| | Gene | GSTP1 |

The genetic information obtained by the assessment tool may thus include genotypes for the genes included in the indicators above.

Table 2, below, lists an exemplary embodiment of the classifications for the Hormone Dominance Category and the associated values of the indicators indicative of each classification. In this example, the values for the indicators are the genotypes of the associated genes.

TABLE 2
Classifications for Hormone Dominance Category

| \Indicators | | | | | |
|---|---|---|---|---|---|
| Classification\ | CYP17A1 | CYP19A1 | SRD5A2 | UGT2B17 | COMT |
| Androgen | AG, GG | CC | GG, GC | 0, 1, 2 | Any |
| Dominance | AA | CC | | 0, 1 | |
| Estrogen | GG, AG | TT, CT | CC | 0, 1, 2 | Any |
| Dominance | | | | | |
| Estrogen- | AA | CT | CC | 1, 2 | AA, |
| Balanced | | | | | AG |
| Androgen- | AA | CT, CC | CC, CG | 0 | Any |
| Balanced | | | | | |
| Co Dominance | GG, AG | TT, CT | GG, GC | 1,0 | Any |

In Table 2 above, each line in a row of values represents a different set of values indicative of the associated classification, letters represent a particular genotype, numbers represent a quantity of copies of a particular gene present, and a blank value or “Any” indicates any genotype may apply. For example, the row for Androgen Dominance includes two separate lines, and thus two sets of values associated with Androgen Dominance whereby: a first set includes AG or GG for CYP17A1, CC for CYP19A1, GG or GC for SRD5A2, 0 or 1 or 2 for UGT2B17, and Any for COMT; and a second set includes AA for CYP17A1, CC for CYP19A1, any for SRD5A2, 0 or 1 for UGT2B17, and Any for COMT.

Table 3, below, lists an exemplary embodiment of the classifications for Risk of Estrogen Toxicity.

TABLE 3
Classifications for Risk of Estrogen Toxicity Category

| \ Indicators | | | |
|---|---|---|---|
| Classification \ | CYP1A1 | CYP1B1 | CYP3A4 |
| Decreased Risk | AG, GG | CC | AA |
| | AA | | |

| | |
|---|---|
| Increased Risk | Anything Outside of Decreased |
| | Risk |

Table 4, below, lists an exemplary embodiment of the classifications for Methylation Detoxification.

TABLE 4
Classifications for Risk of Methylation Detoxification Category

| \ Indicators | | | | | |
|---|---|---|---|---|---|
| Classification \ | MTHFR | SHMT1 | MTRR | MTR | MTHFR-2 |
| OPTIMAL | GG | GG | AA | AA | AA |
| | AG | GG | AA | AA | AA |
| | GG | GG | AA | AA | AG |
| AVERAGE | AG | GG | AA | AA | AG |
| | GG | GG | AG/GG | AA | AA |
| | AA | GG | AA | AA | AA |
| | GG | AG | AA | AA | AA |
| | GG | GG | AA | AG | AA |

| | |
|---|---|
| SUBOPTIMAL | ANYTHING OUTSIDE THE ABOVE |

Table 5, below, lists an exemplary embodiment of the classifications for Glutathionization Detoxification.

TABLE 5
Classifications for Risk of Methylation Glutathionization Category

| \ Indicators | | | |
|---|---|---|---|
| Classification \ | GSTT1 | GSTM1 | GSTP1 |
| OPTIMAL | 2 | 1 | AA |
| | 2 | 0 | AA |
| | 1 | 2 | AA |
| | 1 | 1 | AA |
| | 2 | 1 | GG |
| | 2 | 1 | AG |
| AVERAGE | 2 | 2 | AA, AG, GG |
| | 1 | 2 | AA |
| | 1 | 1 | AA |
| | 2 | 1 | GG |
| | 2 | 1 | AG |
| | 1 | 0 | AA |

| | |
|---|---|
| SUBOPTIMAL | Anything Outside the Above |

The assessment tool may apply one or more exception rules to the classifications above. For example, in a first exception rule for the Glutathionization Detoxification category, if (i) SOD2 has the genotype TT and GPX has the genotype TT, or (ii) if SOD2 has the genotype CC and GPX has the genotype TT, then [1, 0, AA] in table 5 above moves from AVERAGE to SUBOPTIMAL.

In a second exemplary exception rule, certain values of a first additional indicator, listed in Table 6 below, override the classification for Risk of Estrogen Toxicity to be “DECREASED” but also cause the assessment tool to add profile information indicating that risk associated with Birth Control is “LIKELY,” and that Lifelong Estrogen toxicity is “INCREASED.”

TABLE 6
Second Exception Rule for Risk of Estrogen Toxicity Category

| Indicators | CYP17A1 | SRD5A2 | CYP19A1 | UGT2B17 | CYP1B1 | COMT | Methylation Detoxification |
|---|---|---|---|---|---|---|---|
| Values | AA | AG | CC | | AG, GG | GG | Optimal |
| | AG, GG | GG, GC | | 1, 2 | AG, GG | GG | Optimal |

As shown in Table 6 above, the first additional indicator depends on the value of genes as well as the value of the Methylation Detoxification classification, which in this exception rule also acts as an indicator

In a third exemplary exception rule, certain values of a second additional indicator, listed in Table 7 below, override the classification for Risk of Estrogen Toxicity to be “DECREASED” but also cause the assessment tool to add profile information indicating that risk associated with Birth Control is “LIKELY,” and that Lifelong Estrogen toxicity is “INCREASED.”

TABLE 7
Third Exception Rule for Risk of Estrogen Toxicity Category

| Indicators | CYP1B1 | COMT | MTHFR | SHMT1 | GSTT1 | GSTP1 |
|---|---|---|---|---|---|---|
| Values | GG, GC | AG | GG | GG | 1 | AA |

In a fourth exemplary exception rule, certain values of a third additional indicator, listed in Table 8 below, override the classification for Risk of Estrogen Toxicity to be “DECREASED” but also cause the assessment tool to add profile information indicating that risk associated with Birth Control is “LIKELY,” and that Lifelong Estrogen toxicity is “INCREASED.”

TABLE 8
Fourth Exception Rule for Risk of Estrogen Toxicity Category

| | | | Methylation |
|---|---|---|---|
| Indicators | CYP1B1 | COMT | Detoxification |
| Values | AG, GG | GG | OPTIMAL |

As shown in Table 8 above, the third additional indicator depends on the value of genes as well as the value of the Methylation Detoxification classification, which in this exception rule also acts as an indicator.

Based on the indicators and rules above, the assessment tool may determine a classification for each category using the genetic information of the subject. The assessment tool may also obtain profile information and intervention information based on the identified classifications.

For example, profile information associated with a “Co-Dominant” classification for the hormone dominance category may include the following text: “As a co-dominant profile, you will often display aspects of both estrogen and androgen dominance. There will be aspects of your genetic, nutrition, lifestyle and environmental factors that will contribute to the prevalence of which dominance is more apparent. As an individual that leans more towards estrogen dominance, you may experience symptoms such as increased fat deposition, localized cellulite development and/or heavier menstrual cycles. You may also have a curvier figure with larger breast development and wider hips.”

In another example, profile information associated with an “Estrogen Dominant” classification may include the following text: “As an estrogen dominant profile, you are more likely to possess potentially higher than optimal levels of estrogens (estradiol/estrone). During your menstrual years, you are generally more likely to develop larger breasts, wider hips, and experience heavier menstrual cycles and/or uterine fibroids. Despite a healthy diet and lifestyle, you may tend to store fat and develop cellulite (even when at a healthy weight). You may be at an increased risk of estrogen toxicity depending on additional factors like diet, lifestyle and environment. If you display a pronounced estrogen dominant profile, speak to your clinician about dietary, supplemental, and lifestyle solutions that can modulate your estrogen dominance.”

In a further example, profile information associated with an “Androgen Dominant” classification may include the following text: “As an androgen dominant profile, you are more likely to possess potentially higher than optimal levels of androgens. You are more likely to display physical characteristics such as a generally slimmer or slender physique, a resistance to weight gain, and an increased predisposition towards building and retaining lean muscle mass. Cellulite may not be a big concern for you. You are more likely to be predisposed towards increased hair thinning and loss, cystic acne and in some cases, excess facial and body hair. You may have an increased predisposition towards polycystic ovarian syndrome (PCOS). If you display a more pronounced androgen dominant profile, speak to your clinician about dietary, supplemental, and lifestyle solutions that can help balance your androgen dominance.”

In an additional example, profile information associated with an “Estrogen Balanced” classification may include the following text: “An estrogen-balanced profile is generally considered a desirable hormone profile spectrum. With optimal nutrition and lifestyle, and during your menstrual years, you are more likely to attain a fit physique, with the ability to build and retain lean muscle mass. You are also less likely to develop cellulite. You have a typically normal menstrual cycle and can look forward to generally muted menopausal symptoms. Depending on your lifestyle, diet and environment, you may experience some symptoms of estrogen dominance throughout your life—such as increased fat deposition, localized cellulite development and/or heavier menstrual cycles. However, these symptoms will be muted compared to those who are classified as estrogen dominant.”

In another example, profile information associated with an “Androgen Balanced” classification may include the following text: “An androgen-balanced profile is generally considered a desirable hormone profile spectrum. With optimal nutrition and lifestyle, and during your menstrual years, you are more likely to attain a fit physique, with the ability to build and retain lean muscle mass. You are also less likely to develop cellulite. You have a typically normal menstrual cycle and can look forward to generally muted menopausal symptoms. Depending on your lifestyle, diet and environment, you may experience some symptoms of androgen dominance throughout your life—such as a lean or slim figure, increased body hair, irregular menstrual cycles and/or thinning hair. However, these symptoms will be muted compared to those who are classified as androgen dominant.

In some embodiments, the assessment tool may individually identify illnesses or risk factors. For example, Table 9, below, lists an exemplary embodiment of risk factors and corresponding classifications and/or genotypes.

TABLE 9
Risk Factors and Criteria

| Increased Risk | Criteria |
|---|---|
| Premature Ovarian Failure | AG or GG result for the CYP17A1 |
| Irregular Ovulation | Androgen Dominance OR Increased |
| | Risk Estrogen Toxicity BUT NOT both |
| Lifelong Risk of Estrogen | Increased Risk Estrogen Toxicity |
| Toxicity | |
| Endometriosis/Fibrosis | Increased Risk Estrogen Toxicity |
| Polycystic Ovarian Syndrome | Androgen Dominance AND/OR |
| | Estrogen Toxicity |
| Vascular Inflammation | Increased Risk Estrogen Toxicity |
| Neurologic Inflammation | Increased Risk Estrogen Toxicity |

In some embodiments, the assessment tool may identify risk factors associated with a particular medication, supplement, or action. For example, a subject with “SUBOPTIMAL” Methylation Detoxification classification may be associated with a decreased risk associated with vitamin B9 supplementation, and an increased risk associated with vitamin B12 supplementation. It should be understood that these examples are illustrative only, and that any suitable risk factors may be identified.

Intervention information obtained by the assessment tool may include one or more recommendations or directions to enact one or more interventions. For example, for a subject classified with “SUBOPTIMAL” Methylation Detoxification, intervention information may include the following directions: “Avoid a diet rich in inflammatory foods like processed sugars, processed meat, excessive alcohol, refined carbohydrates and artificial trans fats. Avoid inflammatory lifestyle choices like smoking, lack of exercise, and poor sleeping habits. Avoid environments with inflammatory environmental agents—This may include golf courses, which are high in pesticides, older buildings and homes, which may harbor mold, and city centers, which have a higher level of pollution. When considering supplementation, avoid high doses of B vitamins, green tea extract, and polyphenols like quercetin, which can all influence your methylation cycle. Speak to your healthcare provider about what's considered healthy levels of these supplements for your unique profile. Keep in mind that individual ingredients which can otherwise be beneficial, may be harmful at higher doses/different forms for different individuals.”

It should be understood that the categories and classifications above are illustrative only, and that any suitable combination of categories, classifications, indicators, and genes may be used.

### Example 2: Male Subject Hormone Report

A Male Subject Hormone Report may provide a personalized summary of the genomics that may influence reproductive hormone pathways, and consequently, the predisposition with which a subject's body maintains the delicate hormone balance which may impact body type, male health concerns such as gaining and retaining muscle, male-pattern baldness, libido, prostate hyperplasia, and cardiovascular disease, and/or risks associated with hormone replacement. The report may include medical or other intervention recommendations such as, for example, recommendations with regard to diet, environment, and lifestyle.

To develop a Male Subject Hormone Report, an assessment tool, such as the assessment tool described above with regard to FIG. 2, may obtain genetic information for a male subject. The Report may be associated with one or more of the following categories: hormone dominance; low testosterone; male pattern baldness; benign prostate hyperplasia; lean muscle preference; low libido; and risk associated with hormone replacement. Tables 10-19 depict an example of various relationships that may be included in a genotype-based model used by the assessment tool. Table 10 below lists an exemplary embodiment of the indicators associated with each category.

TABLE 10
Categories for Profile Report

| Category | Indicators Type | Indicators |
|---|---|---|
| Hormone | Gene | CYP17A1 |
| Dominance | Gene | CYP19A1 |
| | Gene | SRD5A2 |
| | Gene | UGT2B17 |
| Low T | Gene Group | Hormone |
| | | Dominance |
| | | Level of |
| | Gene Group | Testosterone |
| Male Pattern | Gene Group | Hormone |
| Baldness | | Dominance |
| Benign Prostate | Gene Group | Hormone |
| Hyperplasia | | Dominance |
| | Gene | CYP17A1 |
| | Gene | SRD5A2 |
| Lean Muscle | Gene Group | Hormone |
| Preference | | Dominance |
| | Gene | CYP17A1 |
| | Gene | SRD5A2 |
| | Gene | UGT2B17 |
| Low Libido | Gene Group | Hormone |
| | | Dominance |
| | Gene | CYP17A1 |
| | Gene | SRD5A2 |
| | Gene | UGT2B17 |
| | Gene | CYP19A1 |
| Risk for Hormone | Gene Group | Hormone |
| Replacement | | Dominance |

The genetic information obtained by the assessment tool may thus include genotypes for the genes included in the indicators above.

Table 11, below, lists an exemplary embodiment of the classifications for the Hormone Dominance category and the associated values of the indicators indicative of each classification.

TABLE 11
Classifications for Hormone Dominance Category

| \ Indicators | | | | |
|---|---|---|---|---|
| Classification \ | CYP17A1 | SRD5A2 | CYP19A1 | UGT2B17 |
| Androgen Dominance | GG, AG | GG, CG | CC | 0, 1, 2 |
| | GG, AG | GG, CG | CT | 0, 1 |
| Estrogen Dominance | GG, AG | CC | CT, TT | 0, 1, 2 |
| Estrogen-Non- | AA | CC | CT, TT | 0, 1, 2 |
| Dominant | | | | |
| | AA | GG,CG | CC | 0, 1, 2 |
| Androgen-Non- | AA | GG, CG | CT | 0, 1 |
| Dominant | AA | CG, GG | TT | 0, 1, 2 |
| | AA | CG | CT | 2 |
| | AA | CC | CC | 0, 1, 2 |
| Co Dominance | GG, AG | CG, GG | TT | 0, 1, 2 |
| | GG,AG | CG | CT | 2 |
| | GG,AG | CC | CC | 0, 1, 2 |

Table 12, below, lists an exemplary embodiment of the classifications for Low Testosterone.

TABLE 12
Classifications for Low Testosterone Category

| \ Indicators | | Level of |
|---|---|---|
| Classification \ | Hormone Dominance | Testosterone |
| Increased Risk | Estrogen- Non-Dominant, | Likely |
| | Estrogen Dominance | Decreased |
| Normal Risk | Androgen-Non-Dominant, | |
| | Co Dominance | |
| Decreased Risk | Androgen Dominance | |

Table 13, below, shows an exemplary embodiment of the criteria for the values of the Level of Testosterone indicator.

TABLE 13
Criteria for Level of Testosterone Indicator

| \ Genes | | | | |
|---|---|---|---|---|
| Indicator Value | | DHT | | Metabolization |
| \ | CYP17A1 | levels | Estrogen levels | levels |
| Likely | AA, GG | | | |
| Increased | | | | |
| Likely Normal | AA | | | |
| Likely | AA | Normal, | Normal, | Normal, |
| Decreased | | Increased | Increased | Increased |

Table 14, below, shows an exemplary embodiment of the criteria for the values of the DHT Level indicator.

TABLE 14
Criteria for Level of DHT Indicator

| \ Genes | | |
|---|---|---|
| Indicator Value \ | SRD5A2 | CYP17A1 |
| Likely Increased | CG | AG, GG |
| | GG | AG, GG |
| Likely Normal | CG | |
| | GG | |
| Likely Decreased | CC | |

Table 15, below, shows an exemplary embodiment of the criteria for the values of the Level of E metabolize indicator.

TABLE 15
Criteria for Level of E Metabolize Indicator
\ Genes

| Indicator Value \ | CYP17A1 | CYP19A1 |
|---|---|---|
| Likely Increased | AG, GG | CT |
| | AG, GG | TT |
| Likely Normal | | CT |
| | | TT |
| Likely Decreased | | CC |

Table 16, below, shows an exemplary embodiment of the criteria for the values of the Male Pattern Baldness indicator.

TABLE 16
Criteria for Male Pattern Baldness Indicator

| \ Genes | |
|---|---|
| Indicator Value \ | Hormone Dominance |
| Likely Increased | Androgen Dominance, |
| | Co Dominance |
| Likely Normal | Androgen-Non-Dominant, |
| | Estrogen Dominance |
| Likely Decreased | Estrogen- Non-Dominant |

Table 17, below, shows another exemplary embodiment of the criteria for the values of the Male Pattern Baldness indicator, whereby the value of the indicator is indicative of a magnitude of risk for Male Pattern Baldness, with 1 being the lowest risk, and 5 being the highest risk.

TABLE 17
Criteria for Male Pattern Baldness Risk Magnitude Indicator

| \Genes | | | | | | |
|---|---|---|---|---|---|---|
| Indicator | Hormone | CYP- | | | CYP- | UGT- |
| Value\ | Dominance | 17A1 | SRD5A2 | AR | 19A1 | 2B17 |
| 1 | Estrogen- | | | | | |
| | Non- | | | | | |
| | Dominant | | | | | |
| 2 | Estrogen | GG, AG | | | TT | 1, 2 |
| | Dominance | | | | | |
| 3 | Estrogen | GG, AG | | | CT | 0, 1 |
| | Dominance, | | | | | |
| | Androgen- | | | | | |
| | Non- | | | | | |
| | Dominant | | | | | |
| 4 | Androgen | | | | | |
| | Dominance, | | | | | |
| | Co Dominance | | | | | |
| 5 | | GG, AG | GG | CC | TT | 0 |

Table 18, below, shows an exemplary embodiment of the criteria for the values of the Benign Prostate Hyperplasia indicator.

TABLE 18
Criteria for Benign Prostate Hyperplasia Indicator

| \ Genes | | | Hormone | |
|---|---|---|---|---|
| Indicator Value \ | CYP17A1 | SRD5A2 | Dominance | UGT2B17 |
| Likely Increased | AG, GG | GG, CG | | |
| Likely Normal | | | Androgen Non- | |
| | | | Dominant, Estrogen | |
| | | | Dominance | |
| Likely | | | Estrogen- Non- | |
| Decreased | | | Dominant | |

Table 19, below, shows an exemplary embodiment of the criteria for the values of the Lean Muscle Preference indicator.

TABLE 19
Lean Muscle Preference Indicator

| \ Genes | | | Hormone | |
|---|---|---|---|---|
| Indicator Value \ | CYP17A1 | SRD5A2 | Dominance | UGT2B17 |
| Likely Increased | AG, GG | GG, CG | | |
| Likely Normal | | | | |
| Likely | AA | | Estrogen Non- | 1, 2 |
| Decreased | | | Dominant, | |
| | | | Estrogen | |
| | | | Dominance | |

Table 20, below, shows an exemplary embodiment of the criteria for the values of the Low Libido indicator.

TABLE 20
Criteria for Low Libido Indicator

| \ Genes | | | | |
|---|---|---|---|---|
| Indicator | | | UGT2 | |
| Value \ | SRD5A2 | Hormone Dominance | B17 | CYP19A1 |
| Likely | CC | Estrogen Dominance | 2 | CC, TT |
| Increased | | | | |
| Likely | | Androgen-Non- | | |
| Normal | | Dominant, | | |
| | | Estrogen- Non- | | |
| | | Dominant | | |
| Likely | | Androgen Dominance, | | |
| Decreased | | Co Dominance | | |

Table 21, below, shows an exemplary embodiment of the criteria for the values of the Risk for Hormone Replacement indicator.

TABLE 21
Criteria for Risk for Hormone Replacement Indicator

| \Genes | |
|---|---|
| Indicator Value\ | Hormone Dominance |
| Likely Increased | Androgen Dominance, |
| | Estrogen Dominance, |
| | Co Dominance |
| Likely Normal | Androgen Non-Dominant, |
| | Estrogen Non-Dominant |
| Likely Decreased | |

Based on the indicators and rules above, the assessment tool may employ the genotype-based model to determine a classification for each category using the genetic information of the subject. The assessment tool may also obtain profile information and intervention information based on the identified classifications.

For example, profile information associated with an “Estrogen-Non-Dominant” classification for the hormone dominance category may include the following text related to body type: “Estrogen non-dominant: As a non-dominant male, you will benefit from the best of both worlds (androgens and estrogens) without the excessive characteristics/predispositions of either hormone dominance. With optimal nutrition and lifestyle, you are more likely to attain a healthy physique, with the ability to build and retain lean muscle mass. You are also less likely to develop significant fat deposition. In general, you produce a normal, healthy amount of testosterone that is in balance with your estrogen and DHT production. You are also more likely to keep your hair throughout your life. Keep in mind that the decisions you make for your diet, nutrition and lifestyle can potentially skew you towards estrogenization, which would exaggerate symptoms associated with estrogen dominance. Testosterone/androgen replacement therapy can exaggerate signs of estrogen dominance in males that are already genetically prone to estrogen dominance. In such cases, your clinician might consider co-administering an appropriate aromatase (CYP19A1) inhibitor to avoid unexpected estrogenic side effects such as gynecomastia (male breasts), reduced libido and weight gain. Always consult your clinician about the associated risks prior to initiating a hormone replacement program of any kind.”

Profile information associated with an “Androgen-Non-Dominant” classification for the hormone dominance category may include the following text related to body type: “Androgen non-dominant: As a non-dominant male, you will benefit from the best of both worlds (androgens and estrogens) without the excessive characteristics/predispositions of either hormone dominance. With optimal nutrition and lifestyle, you are more likely to attain a healthy physique, with the ability to build and retain lean muscle mass. You are also less likely to develop significant fat deposition. In general, you produce a normal, healthy amount of testosterone that is in balance with your estrogen and DHT production. You are also more likely to keep your hair throughout your life. Keep in mind that the decisions you make for your diet, nutrition and lifestyle can potentially skew you towards androgenization, which would exaggerate symptoms associated with androgen dominance. Some androgen dominant males may be slim and lean, but have difficulty putting on muscle mass. These males often simultaneously have a faster T and DHT clearance capacity (due to fast UGT genes). These men also find that as they get older and their T and DHT levels decline, so too does their muscle tone. They may stay lean, but they fall into a “use it or lose it” mode with their muscle. NOTE: certain health risks such as BPH and cardiovascular disease can be exaggerated in androgen dominant males who receive androgen hormone replacement.”

Profile information associated with an “Androgen Dominance” classification for the hormone dominance category may include the following text related to body type: “Androgen Dominant: As an androgen dominant male, you are more likely to possess potentially higher than optimal levels of androgens. You are more likely to display physical characteristics such as a generally slimmer or slender physique, a resistance to weight gain, and you may find it easier than most to build and retain lean muscle mass. At the same time, you are more likely to show signs of balding sooner rather than later, develop cystic acne, and in some cases, develop excess facial and body hair. You may also have an increased predisposition towards benign prostate hyperplasia (BPH). If you display a more pronounced androgen dominant profile, speak to your clinician about dietary, supplemental, and lifestyle solutions that can help balance your androgen dominance. Some androgen dominant males may be slim and lean, but have difficulty putting on muscle mass. These males often simultaneously have a faster T and DHT clearance capacity (due to fast UGT genes). These men also find that as they get older and their T and DHT levels decline, so too does their muscle tone. They may stay lean, but they fall into a “use it or lose it” mode with their muscle. NOTE: certain health risks such as BPH and cardiovascular disease can be exaggerated in androgen dominant males who receive androgen hormone replacement.”

Profile information associated with a “Co Dominance” classification for the hormone dominance category may include the following text related to body type: “Co-Dominant: As a co-dominant profile, you will often display characteristics of both estrogen and androgen dominance. The type of dominance that you will tend to lean towards will depend largely on non-genetic factors like nutrition, lifestyle, and environmental factors. Co-dominant males generally tend to lean towards androgenization during their youth and into middle age. However, they may find an increased predisposition towards estrogen dominance in the later years of their life as their androgen production naturally begins to decrease. Some androgen dominant males may be slim and lean, but have difficulty putting on muscle mass. These males often simultaneously have a faster T and DHT clearance capacity (due to fast UGT genes). These men also find that as they get older and their T and DHT levels decline, so too does their muscle tone. They may stay lean, but they fall into a “use it or lose it” mode with their muscle. NOTE: certain health risks such as BPH and cardiovascular disease can be exaggerated in androgen dominant males who receive androgen hormone replacement.”

Profile information associated with an “Estrogen Dominance” classification for the hormone dominance category may include the following text related to body type: “As an Estrogen Dominant male, you are more likely to possess potentially higher than optimal levels of estrogens. You will find it more challenging than normal to put on and retain lean muscle mass. You will likely need to work out for longer and more intense periods versus the average male to increase and retain muscular development. Even when you put on muscle, it may be more difficult for you to display a lean and cut look. You are also more likely to be predisposed to increased fat deposition versus androgen dominant males. However, you are less prone to acne development and have a reduced risk of male pattern baldness. If you display pronounced estrogen dominance, speak to your clinician about dietary, supplemental, and lifestyle solutions that may mitigate your estrogen dominance. Testosterone/androgen replacement therapy can exaggerate signs of estrogen dominance in males that are already genetically prone to estrogen dominance. In such cases, your clinician might consider co-administering an appropriate aromatase (CYP19A1) inhibitor to avoid unexpected estrogenic side effects such as gynecomastia (male breasts), reduced libido and weight gain. Always consult your clinician about the associated risks prior to initiating a hormone replacement program of any kind.”

It should be understood that the information discussed above as included in a genetic profile report is exemplary only, and that any suitable information may be included in a variety of reports.

### Example 3—Weight-based Subject Hormone Report

This example illustrates another exemplary embodiment of a Subject Hormone Report. This example may include aspects that are similar to the previous Examples 1 and 2, with differences discussed in more detail in the following.

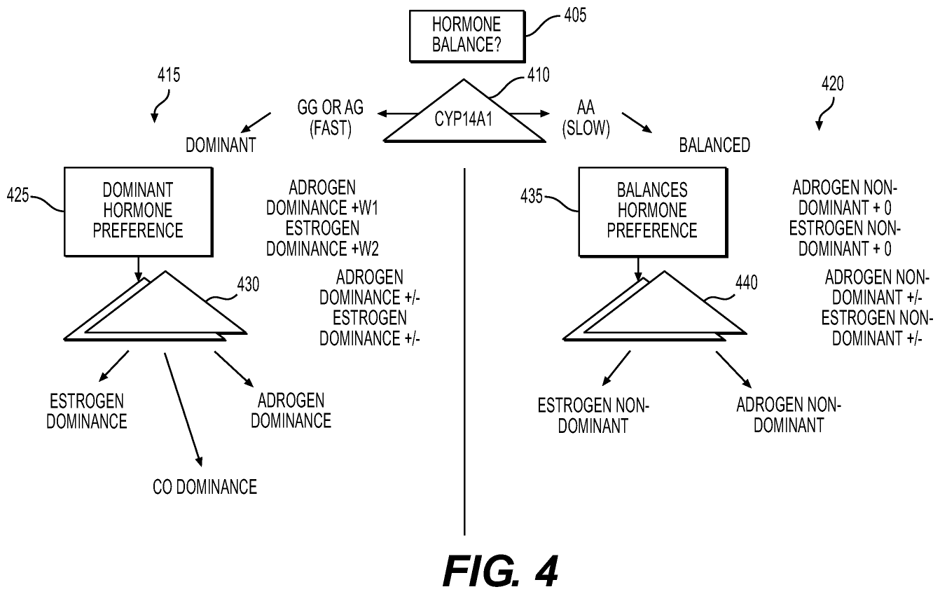

To develop a Weight-based Subject Hormone Report, an assessment tool, such as the assessment tool described above with regard to FIG. 2, may obtain genetic information for a subject, and employ a genotype-based model. FIG. 4 depicts a schematic diagram illustrative of an exemplary process of developing a Weight-based Subject Hormone Report.

The model may include the category of Hormone Balance 405, which may be classified as either “Dominant” or “Balanced.” In this example, the classification for the Hormone Balance category 405 may act as decision tree, or the like. In this example, the classification of the Hormone Balance 405 may be determined based on a first indicator 410 associated with the CYP17A1 gene. For genotypes of GG or AG, this first indicator 410 may have a value of FAST, the Hormone Balance category 405 may be classified as Dominant, and the model may proceed under a first decision tree path 415. For genotypes of AA, the first indicator 410 may have a value of SLOW, the Hormone Balance category 405 may be classified as Balanced, and the model may proceed under a second decision tree path 420.

Under the first path 415, the model includes a Dominant Hormone Preferences sub-category 425, which has possible classifications of Androgen Dominance, Estrogen Dominance, and Co Dominance. The classification of the sub-category 425 may be determined by comparing weights associated with one or more of the classifications. In this example, the classifications of Androgen and Estrogen Dominance are initialized with non-zero and numerically equal weights W1 and W2, respectively. The sub-category 425 may be associated with one or more further indicators 430. Each indicator 430 has one or more possible values, and each possible value may add to or modify the weight of one or more of the classifications.

For example, one of the further indicators 430 may be associated with the CYP19A1 gene, and may have different values depending on the genotype of that gene, e.g., a first value for a first genotype that adds a numerical value M1 to the Androgen Dominance classification, a second value for a second genotype that adds a numerical value M2 to the Estrogen Dominance classification, a third value for a third genotype that adds a numerical value M3 to the Estrogen Dominance classification, etc.

The model may then select a classification for the sub-category based on the weights associated with each possible classification that were initialized and/or set by the further indicators. In one example, the model may sum all of the weights associated with each classification, and select the classification having the highest cumulative weight. In some examples, the model may select a classification based on one or more criteria. For instance, the model may select Co Dominance based on the cumulative weights of Androgen and Estrogen Dominance being equal. In other examples, any suitable comparison of different weights may be used.

Under the second path 420, the model may include a Balanced Hormone Preferences sub-category 435, which may have possible classifications of Androgen Non-Dominant and Estrogen Non-Dominant. The classification of the sub-category 435 may be determined by comparing weights associated with each classification. In this example, each classification is initialized with numerically zero weights. The sub-category 435 may be associated with one or more further indicators 440. Each indicator 440 has one or more possible values, and each possible value may add to or modify the weight of one or more of the classifications.

For example, one of the further indicators 440 may be associated with the CYP19A1 gene, and may have different values depending on the genotype of that gene, e.g., a first value for a first genotype that adds a numerical value M1 to the Androgen Non-Dominant classification, a second value for a second genotype that adds a numerical value M2 to the Estrogen Non-Dominant classification, a third value for a third genotype that adds a numerical value M3 to the Estrogen Non-Dominant classification, etc. In this example, the second path 420 may include one or more indicators also included in the first path 415. In some embodiments, different paths may include different and/or overlapping indicators.

The model may then select a classification for the sub-category based on the weights associated with each possible classification that were initialized and/or set by the further indicators. In one example, the model may sum all of the weights associated with each classification, and select the classification having the highest cumulative weight. In other examples, any suitable comparison of different weights may be used.

For instance, for a subject with a genotype of GG for the CYP17A1 gene, the first indicator 410 may have a value of “FAST,” and thus the genetic model for this subject includes the Hormone Dominance Preference sub-category 425 with possible classifications of Androgen Dominance, Estrogen Dominance, and Co Dominance. Androgen Dominance and Estrogen Dominance may include initialized weights W1 and W2, respectively. Additional weights may add or modify each classification based on values for further indicators 430 based on the subject's genotype. If the subject's genotype results, for example, in a higher cumulative weight for Androgen Dominance, the Hormone Dominance Preference sub-category 425 may be classified as Androgen Dominance.

The model may retrieve profile information and/or intervention information based on the selected classifications, and incorporate such information into a report in a manner similar to the procedures discussed in other examples above.

It should be understood that the categories, classifications, and indicators, above are exemplary only. In various embodiments, categories, classifications, indicators, and genes such as those discussed above in Example 1 for a female subject, or discussed above in Example 2 for a male subject, may be used. Further any suitable weights and/or process for comparing weights of different classifications may be used.

It should be understood that aspects of generating a weight-based report in the manner discussed above may be applied to any suitable category or classification or combination of categories or classifications. Moreover, weight-based techniques discussed above may be combined with other techniques from other embodiments, or vice versa. In other embodiments, other categories, classifications, and/or indicators may act as a decision tree or the like, and a decision tree may have any number of different possible paths.

### Example 4: Subject Environmental Health Profile

Environmental health includes various health aspects determined by physical, chemical, biological, social, and psychosocial factors in the environment. Wellness interventions in environmental health may include assessing, correcting, controlling, and preventing factors in the environment that can potentially adversely affect the health of present and future generations. As used herein, the term “environmental agent” generally encompasses various factors such as, for example, toxins, pollutants, chemicals, etc. that may be present in an environment, as well as any molecule or substance that enters the human body and which has no clear benefit or utility to cellular function, but rather, has the clear potential to negatively impact cellular behavior. Different genotypes may impact how and how efficiently a person's body may deal with environmental agents. Generally, the net effect of environmental agents is associated with inflammation.

An Environmental Health Profile may be associated with one or more categories relevant to the way in which the body interacts with environmental agents such as, for example, Methylation Detoxification, Glutathionization Detoxification, Antioxidation Detoxification, or the like, and may determine classifications for such categories using indicators associated with various genes or groups of genes, such as via the procedures discussed above or others. In an example, indicators associated with the Environmental Health Profile may be associated with genes such as the genes discussed in the other example above and or other genes. In one example, the indicators in the Profile are associated with genes including: GSTT1; GSTM1; SOD2; GPx; DI02; COMT; MTHFR (1); MTHFR (2); SHMT1; FUT2; MTRR; and/or MTR.

A genotype-based model associated with the above may include risk assessments for supplements, illnesses, risk factors, or the like, and in particular an assessment of risk of inflammation from environmental agents. Other risk factors may include, for example, chemical sensitivities for the subject, risk of Fibromyalgia and/or Neuropathy, risk of autoimmune conditions, risk of chronic fatigue syndrome, or the like. The model may assess how environmental agents may impact the subject's hormone balance.

Further, the Profile generated via the model may direct one or more medical or other interventions, such as modifications to diet or nutrition, lifestyle recommendations that avoid or reduce exposure to environmental agents, supplements that mitigate or counteract illnesses such as inflammation, etc.

### Example 5: Subject Executive Function Report

Executive function includes various behavioral aspects that affect the way in which a person interacts with and perceives the world. A genotype-based model for an Executive Function Profile may be associated with one or more categories relevant to mental activity or processes such as, for example, depression, addiction, anxiety, post-traumatic-stress disorder, ADHD or the like, neuroticism, high irritability, difficulty with focus, etc. Other categories may include categories associated with dietary behaviors, such as emotional binging, sugar or caffeine addiction, risk of obesity, or the like. Other categories may also include physical activity behaviors such as a runner's high, difficulty in exercise motivation, poor concussion response, etc. An assessment tool, such as the assessment tool discussed above, may determine classifications for such categories using indicators associated with various genes or groups of genes, such as genes discussed above or others.

In an example, a category of Depression and Addiction may be associated with an indicator for the COMT gene, an indicator for the DRD2 gene, and an indicator for the 5HTTLPR polymorphism. COMT GG may be defined as FAST, and DRD2 AA may be defined as LOW. A FAST version of the COMT gene may result in faster dopamine clearing from the brain, which may reduce the time that a subject may experience pleasure and thus may result in a feeling of “longing” and not being satisfied. A LOW version of the DRD2 gene may result in fewer dopamine receptors on a subject's neurons, which may reduce the intensity of pleasure experience and result in a feeling of needing to do something more exciting to experience real pleasure. A subject with a combination of a FAST COMT and a LOW DRD2 may exhibit reward seeking behavior, e.g., a need for more and more extremes to experience the “hit” or the “rush” of excitement. Without a pleasurable source, such subjects may fall into depression due to being unable to achieve “pleasure,” or may seek out pleasure in negative sources like alcohol, pornography, or drugs. The addition or deletion in a 5HTTLPR polymorphism may result in dysregulated serotonin production. Subjects with this deletion may be more prone to depressive feelings and/or suicidal feelings because of an inability to appropriately prioritize feelings based on urgency or importance.

A category of Binging Addiction may be associated with an indicator for the COMT gene and an indicator for the DRD2 gene. COMT GG may be defined as SLOW, and COMT DRD2 AA may be defined as HIGH. A SLOW version of the COMT gene may result in slow clearing of dopamine from the brain, which may increase the time that a subject may experience pleasure. This may cause a subject to prefer to “savor the moment.” A HIGH version of the DRD2 gene may result in a high density of dopamine receptors on a subject's neurons. This may increase an intensity of pleasure experience. A combination of a SLOW COMT and a HIGH DRD2 may result in a subject that exhibits binging behavior. Such a subject may often have poor control over their instincts when experiencing pleasure, e.g., may overeat a favorite food, may struggle with premature ejaculation, or may display a form of addiction that is not continuous or repetitive but occurs in binging spurts from time to time. Subjects may avoid addictions for weeks or months, but may return to a behavior in response to a trigger, and thus may have binging periods from time to time.

A category of Anxiety may be associated with an indicator for COMT gene, and an indicator for the ADRA2B gene that is based on additions or deletions of that gene. SLOW COMT combined with ADRA2B Deletion (ID or DD), may result in increased prevalence. A deletion in the ADRA2B gene may result in a receptor that binds to noradrenaline may stay active for a longer period of time. This may result in hypersensitivity. A subject may be more likely to be careful of an activity due to a past negative experience. This may continue for a while after the actual event happened, due to the prolonged sensitivity to noradrenaline, e.g., “the fight or flight response.” Just as SLOW COMT may result in a predisposition towards “high” peaks of pleasures, it may also result in extreme “low” valleys of negative emotions. Subjects may have a tougher time coming out of a ruts, or tend to worry excessively and often replay events in their brain about hypothetical situations before or after an emotional or important event. The addition of a deletion in the ADRA2B gene may result in an increase in emotional attachment. A subject may be more likely to have a stronger and lengthier negative response to emotional events, particularly negative ones. Such individuals may be good at picking out those who are upset in a crowd. They may read other people's actions as a response to a shortcoming within themselves.

A category of Post Traumatic Stress Disorder (“PTSD”) may be associated with an indicator for the ADRA2B gene. ADRA2B Deletions may result in a stronger risk of PTSD due to an increase in attachment of emotions to negative memories. Subjects may be more likely to suffer from PTSD after witnessing or experiencing traumatic events.

A category for Attention Deficit Hyperactive Disorder (“ADHD”) symptoms or the like may be associated with an indicator for the COMT gene and an indicator for the DRD2 gene. A combination of a FAST COMT and a HIGH DRD2 may result in a subject that exhibits ADHD like behavior. Such individuals often bounce from one activity to the next and can easily become bored if they are not challenged.

A category of Neuroticism may be associated with the BDNF gene. BDNF AG or AA is defined as LOW, and may result in increased prevalence. The BDNF gene is associated with improved neuronal health and malleability. It may promote building new pathways for neurons to help the brain deal with difficult or complex situations. A subject with LOW BDNF may exhibit “hamster wheel” behavior —constantly repeating thoughts and finding it difficult to discover unique solutions to problems. Such individuals are usually set in their ways and find change difficult to deal with. They may be more sensitive to social cues, and may have difficulty gaining restful sleep, which is when most BDNF production occurs. They may be very peculiar about the way they present themselves or are represented to the public.

Categories of High Irritability or Difficulty in Focus may be associated with the 5HTTLPR polymorphism of the SLC6A4 gene. Subjects with a deletion in their 5HTTLPR polymorphism may have dysregulated serotonin production. Serotonin controls the response mechanism of humans. A dysregulated serotonin response may result in easy irritation with things that others would normally ignore. Subjects with dysregulated serotonin may also have a hard time maintaining focus in the presence of distractions because of a reduced ability to compartmentalize stimuli based on priority. A subject with dysregulated serotonin may find it difficult to focus on a task once they become aware of a distraction.

The category of Food as Coping Mechanism may be associated with indicators for the COMT, ADRA2B, and SLC6A4 genes, e.g., increased prevalence with COMT AA, ADRA2B ID or DD, and 5HTTLPR LS or SS. Subjects with increased risk of anxiety combined with a deletion in the 5HTTLPR polymorphism of the SLC6A4 gene may be more likely to seek out food as a coping mechanism, particularly if it was tied with an emotional event in the past.

A category for Food and/or Sugar addiction may be associated with an indicator for the Depression category and an indicator for the Binging category. Studies have suggested a significant influence of sugar on neurotransmitters like dopamine. While lifestyle and environment may play a role, sugar's ability to stimulate excessive dopamine release may be a strong contributing factor to addiction, particularly in those with addiction/binging/depressive genotypes.

A category for Caffeine Addiction may be associated with an indicator for the BDNF gene and the 5HTTLPR polymorphism of the SLC6A4 gene. Subjects with the T allele of BDNF may be more likely to have a positive reinforcement to coffee consumption due to the potential BDNF-boosting capabilities of caffeine. Those with dysregulated serotonin reuptake may also be likely to desire coffee for its neuromodulating properties—helping one relax and focus during times of irritability and stimulation.

A category for Obesity may be associated with an indicator for the Addiction category, the Binging category, the BDNF gene, the 5HTTLPR polymorphism, or others. Obesity is a multifactorial metabolic disorder that may involve a detailed evaluation of a person's diet, lifestyle, environment, and genetics. There are several genes associated with an increased risk in Obesity. An emotional aspect of Obesity may be influenced by prefrontal cortex activity, which is influenced by the COMT gene. The BDNF gene may play a role in influencing common habitual eating behaviors, while the 5HTTLPR polymorphism modulates the serotonin response. When combined together, these genes may play a significant role in the onset and prevalence of obesity in conjunction with lifestyle, diet, and environment.

A category of a Runner's High may be associated with an indicator for the BDNF gene, e.g., an increase in prevalence with BDNF A. Exercise has been associated with an increase in BDNF production. A subject with low BDNF production may experience a noticeable elevation in mood while engaging in exercises such as running—e.g., a Runner's High. This noticeable elevation in mood may last for a period after exercise as well.

A category for Exercise Motivation may be associated with an indicator for the COMT gene, and an indicator for the DRD2 gene, e.g., increased prevalence with COMT GG combined with DRD2 GG. Subjects with this genetic combination may find an increased difficulty in maintaining an exercise routine, particularly when they hit a plateau and cannot see visible benefit.

A category for Concussion Response Risk may be associated with the BDNF gene, e.g., increased prevalence with BDNF A. Trauma in the brain can occur through physical, mental, or chemical means. Individuals with lower BDNF expression may have a difficult time repairing or growing new neuronal connections after they are destroyed due to a traumatic experience. As a result, they may be at a greater risk of experiencing further complications (including concussions) in subsequent traumatic occurrences.

### Example 6: Disease Risk Report

A Disease Risk Report may provide personalized assessments for one or more category including a risk of contracting a particular disease, a risk of complications resulting from the disease if contracted, or a risk of symptom severity while ill with the disease. In such a Report, each category may be classified via a risk rating, e.g., an A rating for a lowest risk, B for a low risk, C for a medium risk, D for a high risk, etc. Any suitable rating system may be used. And, each rating may be based on the value(s) of one or more indicators and/or classifications for sub-categories. This type of Report may be used to assess risk for any one of a variety of diseases. As an illustrative example, this Report will be described in further detail with regard to COVID-19.

In an example, the category of risk of complications resulting from having experienced COVID-19 may be classified with a risk rating based on one or more indicators associated with one or more non-genetic factors. For instance, one indicator may increase or decrease a risk score based on a history or lack thereof of hypertension, diabetes, cardiovascular disease, etc. Another indicator may increase or decrease the risk score based on age or age group, gender, body-mass index, waist-to-height ratio, etc., or combinations thereof. A further indicator for hypertension or cardiovascular risk may be based on the history for such conditions, such as in the indicator discussed above, and/or may be based on medical information such as a most-recent test or lab result. In an example, an indicator for hypertension may have a default value in response to a history of hypertension, but if there is not such history, may have a value based on a recent blood pressure test. The values of such indicators may be binary, e.g., a positive or negative response, and the rating for the category may be based on a summation of the response, e.g., a positive response acting as a numerical+1 and a negative response acting as a numerical −1 to arrive at a total score or rating. The values of the indicators may also be numerical weights, genotypes, etc.

In another example, a category of risk of infection by COVID-19 may be based on one or more genetic indicators. For instance, indicators associated with one or more genes may each have a value of either positive or negative, and the risk rating for infection may be based on the total number of positive values. Genes that may be associated with the risk of infection by COVID-19 may include, for example, ACE2 rs1996225, ACE2 rs2285666, or TMPRSS2 rs2070788.

In a further example, a category of risk of symptom severity while ill with COVID-19 may include one or more sub-categories, each of which may include a classification in the form of a risk rating. Sub-categories may include, for example: Vitamin D Activation, Transport, Uptake with indicators associated with the CYP2R1, VDBP/GC, and/or VDR genes; Hypertension and Insulin Resistance with indicators associated with the ACE, CLOCK, TCF7L2, CRY1, DI02, and SLC30A8 genes, Antioxidation and Phase II Detox with indicators associated with the GSTT1, GSTM1, GSTP1, and SLC23A1 genes, Mitochondrial Redox Reaction with indicators associated with the SOD2 and GPX genes, etc. Other categories may include Methylation & Anti-Inflammation, Vascular Inflammation, Mood and Behavior, Addiction, Anxiety, Depression, etc.

The genetic-based model may generate a report for the subject based on the classifications and/or risk ratings. The report may include profile and/or intervention information associated with the classifications and/or risk ratings in a manner similar to reports discussed in other examples. In particular, the report may include one or more intervention recommendations in order to reduce or mitigate a risk of contracting COVID-19, experiencing severe symptoms if contracted, and minimizing a risk of complications.

### Example 7: Cardiovascular Health Report

A Cardiovascular Health Report may provide information associated with various aspects of cardiovascular health and related conditions. An overall health assessment category may be based on the classifications of sub-categories such as, for example, a first category of Endothelium Response, a second category of Metabilization of low-density lipoproteins, a third category of Methylation, a fourth category of Detoxification. The first category of Endothelium Response may be associated with an indicator for the 9P21 gene. The second category of Metabilization of low-density lipoproteins may be associated with an indicator for the APOE gene. Methylation may be associated with indicators for the FUT2, MTHFR, SHMT1, MTRR, MTR, and MTHFR-2 genes. Detoxification may be associated with indicators for the GSTT1, GSTM1, GSTP1, SOD, and COMT genes. Each sub-category may be classified as optimal, average, or suboptimal. The overall health assessment category may be classified as optimal, average-high, average-low, or suboptimal based on, for example, how many of each of the foregoing sub-categories are classified as optimal, suboptimal or average, as shown in Table 22 below.

TABLE 22
Classification of Overall Health Assessment Category

| Classification | Sub-Category Ratings | | |
|---|---|---|---|
| OPTIMAL | 4 optimal | 3 optimal | 3 optimal 1 |
| | | 1 average | suboptimal |
| AVERAGE (HIGH) | 4 average | 3 average | 2 optimal 2 |
| | | 1 optimal | average |
| AVERAGE (LOW) | 3 average 1 | 2 suboptimal | 2 average, 1 optimal, |
| | suboptimal | 2 average | 1 suboptimal |
| SUBOPTIMAL | 4 suboptimal | 3 suboptimal | 3 suboptimal 1 |
| | | 1 average | optimal |

### Example 8: Inflammation Assessment

An inflammation risk assessment may provide an indication of how at risk an individual may be to complications of, and/or an increased severity of, inflammation. The inflammation category (e.g., risk level) may be based on classifications of sub-categories such as detoxification performance, methylation performance, antioxidation performance, and/or endothelium performance.

Detoxification performance may be associated with GSTT1, GSTM1, and GSTP1 as listed in Table 23 below, and may be associated with a grade or weight value based on the genotypes of those genes.

TABLE 23
Classification of Detoxification Performance

| GSTT1 | GSTM1 | GSTP1 | GRADE | POINTS |
|---|---|---|---|---|
| 2 | 2 | AA | A | 2 |
| 2 | 1 | AA | A | 2 |
| 2 | 0 | AA | A | 2 |
| 2 | 1 | AG/GG | B | 1 |
| 2 | 0 | AG/GG | B | 1 |
| 1 | 1 | AA | B | 1 |
| 1 | 0 | AA | B | 1 |
| 1 | 1 | AG/GG | B | 1 |
| 1 | 0 | AG/GG | C | −1 |
| 0 | 1 | AA | C | −1 |
| 0 | 0 | AA | D | −2 |
| 0 | 1 | AG/GG | D | −2 |
| 0 | 0 | AG/GG | D | −2 |

Methylation performance may be associated with MTHFR, SHMT1, MTRR, MTR, and MTHFR-2 as listed in Table 24 below, and may be associated with a grade or weight value based on the genotypes of those genes.

TABLE 24
Classification of Methylation Performance

| MTHFR | SHMT1 | MTRR | MTR | MTHFR-2 | Grade | Points |
|---|---|---|---|---|---|---|
| GG | GG | AA | AA | AA | A | 2 |
| AG | GG | AA | AA | AA | A | 2 |
| GG | GG | AA | AA | AG | A | 2 |
| AG | GG | AA | AA | AG | B | 1 |
| GG | GG | AG/GG | AA | AA | B | 1 |
| AA | GG | AA | AA | AA | B | 1 |
| GG | AG | AA | AA | AA | B | 1 |
| GG | GG | AA | AG | AA | C | −1 |

| | | |
|---|---|---|
| Anything else | D | −2 |

Antioxidation performance may be associated with SOD2 and GPX as listed in Table 25 below, and may be associated with a grade or weight value based on the genotypes of those genes.

TABLE 25
Classification of Antioxidation Performance

| SOD2 | GPX | GRADE | Points |
|---|---|---|---|
| CT | CT | A | 2 |
| CC | CC | B | 1 |
| CC | CT | B | 1 |
| any other combo | | | −1 |

Endothelium performance may be associated with 9P21, 1P21, and PCSK9 as listed in Table 26 below, and may be associated with a respective grade or weight value for the genotypes of each of those genes as sub-sub-categories.

TABLE 26
Classification of Endothelium Performance

| 9P21 | Grade | Points | 1P21 | Grade | Points | PCSK9 | Grade | Points |
|---|---|---|---|---|---|---|---|---|
| 0G | A | 2 | GG | A | 2 | TT | A | 2 |
| 1G | A | 2 | AG | B | 1 | CT | B | 1 |
| 2G | B | 1 | AA | C | 0 | CC | C | 0 |
| 3G | C | −1 | | | | | | |
| 4G | D | −2 | | | | | | |
| 5G | D | −2 | | | | | | |
| 6G | D | −2 | | | | | | |

An overall inflammation risk assessment may be based on a sum of the weights or grades of the categories above, e.g., as listed below in Table 27

TABLE 27
Inflammation Risk Assessment

| Lowest Risk | Low Risk | High Risk | Highest Risk |
|---|---|---|---|
| 8+ points | 3-8 points | 2 to −2 points | less than −2 points |

### Example 9: Cholesterol Risk Assessment

A cholesterol risk assessment may provide an indication of how at risk an individual may be to complications of, and/or an increased prevalence of, high cholesterol. The cholesterol category (e.g., risk level) may be based on classifications of sub-categories such as detoxification performance, methylation performance, antioxidation performance, e.g., using the same or similar weights and grades listed above for example 8, and/or a cholesterol management category.

The cholesterol management category may be based on APOE, as listed in Table 28 below.

TABLE 28
Cholesterol Management

| APOE | Grade | Points | Special Note |
|---|---|---|---|
| (2/2) | C | 0 | Potential Risk of |
| | | | Type 3 HLP |
| (2/3) | A | 2 | |
| (2/4) | B | 1 | |
| (3/3) | B | 1 | |
| (3/4) | C | −1 | |
| (4/4) | D | −2 | |

The overall cholesterol risk assessment may be based on the sum of the weights or grades of the foregoing categories in a similar manner as in example 8, e.g., 8+ points for lowest risk, 3-8 points for low risk, etc.

### Example 10: Hypertension Risk Assessment

A hypertension risk assessment may provide an indication of how at risk an individual may be to complications of, and/or an increased severity of, hypertension. The hypertension category (e.g., risk level) may be based on classifications of sub-categories such as detoxification performance, methylation performance, antioxidation performance, e.g., using the same or similar weights and grades listed above for example 8, and/or a blood pressure management category.

The blood pressure management category may be based on ACE and NOS3, as listed in Table 29 below.

TABLE 29
Blood Pressure Management

| ACE | NOS3 | Grade | Points |
|---|---|---|---|
| AA | GG | A | 2 |
| AA | GT/TT | B | 1 |
| AG | GG | B | 1 |
| AG | GT/TT | C | −1 |
| GG | GG | C | −1 |
| GG | GT/TT | D | −2 |

The overall hypertension risk assessment may be based on the sum of the weights or grades of the foregoing categories in a similar manner as in example 8, e.g., 8+ points for lowest risk, 3-8 points for low risk, etc.

### Example 11: Sleep Assessment

A sleep assessment may evaluate the quality of a person's sleep. The sleep assessment category may be based on a first category of circadian rhythm assessment and a second category of pleasure response assessment.

Table 30 below shows a collection of indicators for circadian rhythm that may each be assigned a grade, weight, or point value based on their respective values.

TABLE 30
Indicators for Circadian Rhythm

| | | | GRADE C- | GRADE D- |
|---|---|---|---|---|
| | GRADE A- | GRADE B- | MINUS 1 | MINUS 2 |
| | 2 POINTS | 1 POINT | POINT | POINTS |
| CLOCK | TT | | CT | CC |
| grade | | | | |
| BDNF | GG | | AG | AA |
| grade | | | | |
| Vitamin | All 3 of the | 2 out of 3 of | 1 out of 3 of | 0 out of 3 of |
| D grade | below | the below | the below | the below |
| CYP2R1 | AA | AA | AA | AA |
| GC | CC | CC | CC | CC |
| VDP | CC | CC | CC | CC |

In the table above, the three genes CYP2R1, GC, and VDP are sub-categories that are used to determine the grade/weight of the Vitamin D category. In other words, table 30 above includes 3 indicators, an indicator for the CLOCK gene, an indicator for the BDNF gene, and an indicator for Vitamin D response, which is itself based on the three genes CYP2R1, GC, and VDP0. Each indicator in table 30 may receive a respective grade.

A summation of the weights/points above may be used to determine an overall circadian rhythm weight or grade, e.g., as listed in table 31 below.

TABLE 31
Overall Circadian Rhythm Grade
CIRCADIAN RHYTHMS GRADES BASED ON GENES

| GRADE A | GRADE B | GRADE C | GRADE D |
|---|---|---|---|
| 5 to 6 | 1 to 4 | minus 3 to | less than minus |
| points | points | 0 points | 3 points |

From the overall circadian rhythm grade, a circadian rhythm weight for the overall sleep assessment may be determined, e.g., as listed in table 32 below.

TABLE 32
Circadian Rhythm Weight
CIRCADIAN RHYTHMS

| GRADE | points towards overall sleep grade |
|---|---|
| A | 2 |
| B | 1 |
| C | −1 |
| D | −2 |

Table 33 below shows a collection of indicators for pleasure response that may each be assigned a grade, weight, or point value based on their respective values in a manner similar to table 30 above.

TABLE 33
Indicators for Pleasure Response

| | | | GRADE C- | GRADE D- |
|---|---|---|---|---|
| | GRADE A- | GRADE B- | MINUS 1 | MINUS 2 |
| | 2 POINTS | 1 POINT | POINT | POINTS |
| COMT | TT | | CT | CC |
| BDNF | GG | | AG | AA |
| grade | | | | |
| Vitamin | All 3 of the | 2 out of 3 of | 1 out of 3 of | 0 out of 3 of |
| D grade | below | the below | the below | the below |
| CYP2R1 | AA | AA | AA | AA |
| GC | CC | CC | CC | CC |
| VDP | CC | CC | CC | CC |

The summation of the weights/points above may be used to determine an overall pleasure response weight or grade, e.g., as listed in table 34 below.

TABLE 34
Overall Pleasure Response Grade
PLEASURE RESPONSE GRADES BASED ON GENES

| GRADE A | GRADE B | GRADE C | GRADE D |
|---|---|---|---|
| 5 to 6 | 1 to 4 | minus 3 to | less than minus |
| points | points | 0 points | 3 points |

From the overall pleasure response grade, a pleasure response weight for the overall sleep assessment may be determined, e.g., as listed in table 35 below.

TABLE 35
Pleasure Response Weight
PLEASURE RESPONSE

| GRADE | points towards overall sleep grade |
|---|---|
| A | 2 |
| B | 1 |
| C | −1 |
| D | −2 |

A combination of the circadian rhythm weight and pleasure response weight may then be used to determine an overall sleep assessment, with a higher combined weight being associated with a higher grade for overall sleep.

### Example 12: Immunity Assessment

An immunity assessment may provide an indication of the performance of a person's immune system. The immunity category (e.g., performance level) may be based on classifications of sub-categories such as vitamin D grade, detoxification performance, methylation performance, e.g., using the same or similar weights and grades listed in one or more of the examples above, and/or a mitochondrial redox category.

The mitochondrial redox category may be based on SOD2 and GPX, as listed in Table 36 below.

TABLE 36
Mitochondrial Redox

| SOD2 | GPX | GRADE | Points |
|---|---|---|---|
| CT | CT | A | 2 |
| CC | CC | B | 1 |
| CC | CT | B | 1 |
| any other combo | | | −1 |

The overall immunity assessment may be based on the weights or grades of the foregoing categories in a similar manner as in one or more of the examples above, e.g., 6-10 points for highest grade, lowest grade for −2 points or less, etc.

It should be understood that the examples provided above are illustrative only, and that one or more aspects of this disclosure may be applied to any suitable assessment, risk determination, or the like. It should be understood that one or more categories, classifications, indicators, genes, criteria, scoring or rating techniques, or other aspects from the examples and discussion above may be used and/or combined for various operations such as generating reports, profiles, intervention recommendations, or the like. The various examples and embodiments above are illustrative only, and are not limiting as to how aspects of this disclosure may be generated, used, or etc.

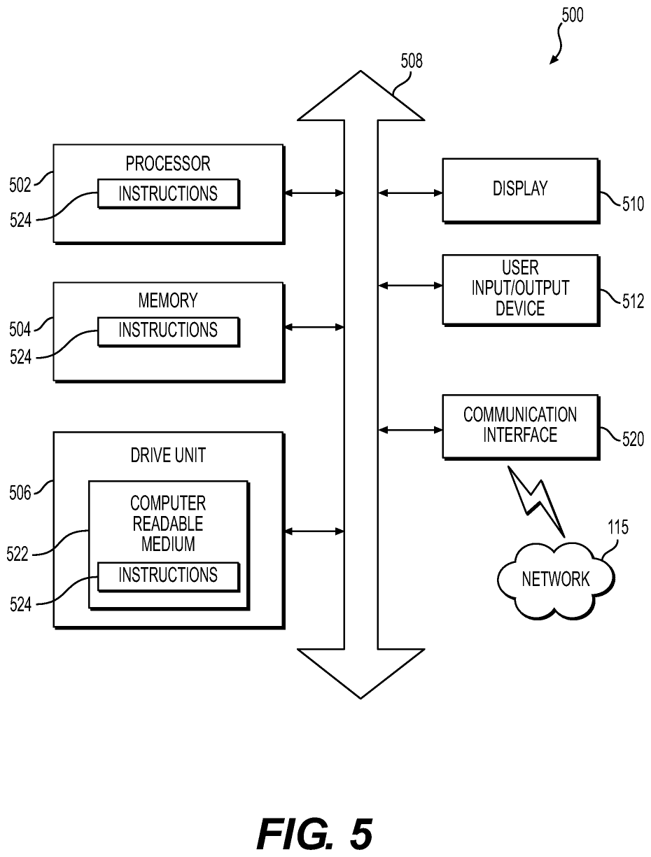

FIG. 5 is a simplified functional block diagram of a computer 500 that may be configured as a device for executing the methods and processes of FIGS. 2-4, according to exemplary embodiments of the present disclosure. The computer 500 may be configured as the profiler system 120 according to exemplary embodiments of the present disclosure. Specifically, in one embodiment, any of the mobile devices, systems, servers, etc., discussed herein may be an assembly of hardware including, for example, a data communication interface 520 for packet data communication. The platform also may include a central processing unit (“CPU”) 502, in the form of one or more processors, for executing program instructions. The platform may include an internal communication bus 508, and a storage unit 506 (such as ROM, HDD, SDD, etc.) that may store data on a computer readable medium 522, although the system 500 may receive programming and data via network communications. The system 500 may also have a memory 504 (such as RAM) storing instructions 524 for executing techniques presented herein, although the instructions 524 may be stored temporarily or permanently within other modules of system 500 (e.g., processor 502 and/or computer readable medium 522). The system 500 also may include input and output ports5 and/or a display 510 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the presently disclosed methods, devices, and systems are described with exemplary reference to transmitting data, it should be appreciated that the presently disclosed embodiments may be applicable to any environment, such as a desktop or laptop computer, an automobile entertainment system, a home entertainment system, etc. Also, the presently disclosed embodiments may be applicable to any type of Internet protocol.

Other embodiments of the disclosure will be apparent to those skilled in the art from consideration of the specification and practice of the disclosure disclosed herein. It is intended that the specification and examples be considered as exemplary only.

In general, any process discussed in this disclosure that is understood to be performable by a computer may be performed by one or more processors. Such processes include, but are not limited to, the processes shown in FIGS. 2 and 3, and the associated language of the specification. The one or more processors may be configured to perform such processes by having access to instructions (computer-readable code) that, when executed by the one or more processors, cause the one or more processors to perform the processes. The one or more processors may be part of a computer system (e.g., one of the computer systems discussed above) that further includes a memory storing the instructions. The instructions also may be stored on a non-transitory computer-readable medium. The non-transitory computer-readable medium may be separate from any processor. Examples of non-transitory computer-readable media include solid-state memories, optical media, and magnetic media.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the disclosed invention requires more features than are expressly recited in any particular embodiment or example. Rather, inventive aspects lie in less than all features of a single foregoing disclosed embodiment. Furthermore, while some embodiments described herein include some but not other features included in other embodiments, combinations of features of different embodiments are meant to be within the scope of the invention, and form different embodiments, as would be understood by those skilled in the art.

Thus, while certain embodiments have been described, those skilled in the art will recognize that other and further modifications may be made thereto without departing from the spirit of the invention, and it is intended to include all such changes and modifications as falling within the scope of this disclosure. For example, functionality may be added or deleted from the block diagrams and operations may be interchanged among functional blocks. Steps may be added or deleted to methods described within the scope of the present disclosure.

1. A computer-implemented method for providing wellness intervention information to a subject, comprising:
receiving, via a processor of a user device associated with a user, genetic information of the subject, the genetic information including a plurality of sets, and each set comprising one or more genes;
assigning, via the processor, a value to each respective set;
assigning, via the processor, a respective classification to the subject in one or more categories, wherein:
each category corresponds to at least one set; and
the assignment of the respective classification is based on the respective values assigned to the at least one corresponding set;
obtaining, via the processor, health profile information and wellness intervention information associated with the assigned classifications of the one or more categories; and
outputting, via the processor, the health profile information and the wellness intervention information to the user via the user device.

2. The computer-implemented method of claim 1, wherein the plurality of sets includes at least one first set that comprises a single gene and at least one second set that comprises two or more genes.

3. The computer-implemented method of claim 1, wherein:
the plurality of sets includes at least one first set and at least one second set; and
one or more of:
the value assigned to the second set depends on the value assigned to the first set; or
the one or more categories includes a first category and a second category, the respective classification assigned to the first category is based on the value assigned to the first set, and the respective classification assigned to the second category is based on the value assigned to the second set and the classification assigned to the first category.

4. (canceled)

5. The computer-implemented method of claim 1, wherein:
the one or more categories includes hormone dominance; and
the at least one set comprises:
one or more of CYP17A1, CYP19A1, SRD5A2, UGT2B15, UGT2B17, CYP1A1, CYP1A2, CYP1B1, CYP3A4, COMT, SOD2, or GPx;
two or more genes chosen from MTHFR, SHMT1, MTRR, MTR, MTHFR-2, and combinations thereof; or
two or more genes chosen from GSTT1, GSTM1, or GSTP1, and combinations thereof.

6. The computer-implemented method of claim 1, wherein the value for at least one of the sets is one or more of a genotype or is assigned based on at least one genotype of the set.

7. (canceled)

8. The computer-implemented method of claim 1, wherein values of sets associated with a respective category are numerical values weighted based on genotypes of the sets, and a combination of the numerical values is associated with one or more possible classifications for the respective category.

9. The computer-implemented method of claim 1, wherein:
at least one of the one or more categories is a decision tree category; and
the method further comprises adding or removing a category, via the processor, to or from the one or more categories based on the respective classification assigned to the decision-tree category.

10. The computer-implemented method of claim 1, wherein the respective classification includes one or more of a rating or risk assessment for the corresponding category.

11. The computer-implemented method of claim 1, further comprising:
directing the subject, via the processor, to enact at least one intervention based on the wellness intervention information.

12-14. (canceled)

15. The computer-implemented method of claim 1, wherein:
assigning the respective classification includes predicting a health state of the subject based on the values assigned to the plurality of sets, and the health profile information includes the predicted health state; and
the predicted health state includes:
a risk of one or more of irregular ovulation, premature ovarian failure, lifelong risk of estrogen toxicity, polycystic ovarian syndrome, endometriosis, vascular inflammation, fibromyalgia, or breast cancer; or
a risk of one or more of low testosterone, male pattern baldness, benign prostate hyperplasia, ability to develop lean muscle, low libido, or complications from hormone replacement therapy.

16-18. (canceled)

19. A system for providing intervention information to a subject, comprising:
a processor; and
a memory operatively connected to the processor and storing instructions that, when executed by the processor, cause the processor to perform acts including:
receiving, via the processor, genetic information of the subject, the genetic information including a plurality of sets, and each set comprising one or more genes;
assigning, via the processor, a value to each respective set;
assigning, via the processor, a respective classification to the subject in one or more categories, wherein:
each category corresponds to at least one set; and
the assignment of the respective classification is based on the respective values assigned to the at least one corresponding set;
obtaining, via the processor, health profile information and wellness intervention information associated with the assigned classifications of the one or more categories; and
outputting, via the processor, the health profile information and the wellness intervention information to a user.

20. The system of claim 19, wherein the plurality of sets includes at least one first set that comprises a single gene and at least one second set that comprises two or more genes.

21. The system of claim 19, wherein:
the plurality of sets includes at least one first set and at least one second set; and
one or more of:
the value assigned to the second set depends on the value assigned to the first set; or
the one or more categories includes a first category and a second category, the respective classification assigned to the first category is based on the value assigned to the first set, and the respective classification assigned to the second category is based on the value assigned to the second set and the classification assigned to the first category.

22. (canceled)

23. The system of claim 19, wherein:
the one or more categories includes hormone dominance; and
the at least one set comprises:
one or more of CYP17A1, CYP19A1, SRD5A2, UGT2B15, UGT2B17, CYP1A1, CYP1A2, CYP1B1, CYP3A4, COMT, SOD2, or GPx;
two or more genes chosen from MTHFR, SHMT1, MTRR, MTR, MTHFR-2, and combinations thereof; or
two or more genes chosen from GSTT1, GSTM1, or GSTP1, and combinations thereof.

24. The system of claim 19, wherein the value for at least one of the sets is one or more of a genotype or is assigned based on at least one genotype of the set e.

25. (canceled)

26. The system of claim 19, wherein values of sets associated with a respective category are numerical values weighted based on genotypes of the sets, and a combination of the numerical values is associated with one or more possible classifications for the respective category.

27. The system of claim 19, wherein:
at least one of the one or more categories is a decision tree category; and
the acts further include adding or removing a category, via the processor, to or from the one or more categories based on the respective classification assigned to the decision-tree category.

28. The system of claim 19, wherein the respective classification includes one or more of a rating or risk assessment for the corresponding category.

29. The system of claim 19, further comprising:
directing the subject, via the processor, to enact at least one intervention based on the wellness intervention information.

30-32. (canceled)

33. The system of claim 19, wherein:
assigning the respective classification includes predicting a health state of the subject based on the values assigned to the plurality of sets, and the health profile information includes the predicted health state; and
the predicted health state includes:
a risk of one or more of irregular ovulation, premature ovarian failure, lifelong risk of estrogen toxicity, polycystic ovarian syndrome, endometriosis, vascular inflammation, fibromyalgia, or breast cancer; or
a risk of one or more of low testosterone, male pattern baldness, benign prostate hyperplasia, ability to develop lean muscle, low libido, or complications from hormone replacement therapy.

34-36. (canceled)

37. A method of predicting a health state of a subject, comprising:
receiving genetic information of the subject, the genetic information including a plurality of sets, and each set comprising one or more genes;
assigning a value to each respective set;
assigning a respective classification to the subject in one or more categories, wherein:
each category corresponds to at least one set;
the assignment of the respective classification is based on the respective values assigned to the at least one corresponding set; and
the assignment of the respective classification is predictive of the health state of the subject;
generating a health profile of the subject based on the predicted health state from the assigned classifications for the one or more categories; and
providing the health profile of the subject to the subject.

38. The method of claim 37, wherein the plurality of sets includes at least one first set that comprises a single gene and at least one second set that comprises two or more genes.

39. The method of claim 37, wherein:
the plurality of sets includes at least one first set and at least one second set; and
one or more of:
the value assigned to the second set depends on the value assigned to the first set; or
the one or more categories includes a first category and a second category, the respective classification assigned to the first category is based on the value assigned to the first set, and the respective classification assigned to the second category is based on the value assigned to the second set and the classification assigned to the first category.

40. (canceled)

41. The method of claim 37, wherein:
the one or more categories includes hormone dominance; and
the at least one set comprises:
one or more of CYP17A1, CYP19A1, SRD5A2, UGT2B15, UGT2B17, CYP1A1, CYP1A2, CYP1B1, CYP3A4, COMT, SOD2, or GPx;
two or more genes chosen from MTHFR, SHMT1, MTRR, MTR, MTHFR-2, and combinations thereof; or
two or more genes chosen from GSTT1, GSTM1, or GSTP1, and combinations thereof.

42. The method of claim 37, wherein the value for at least one of the sets is one or more of a genotype or is assigned based on at least one genotype of the set.

43. (canceled)

44. The method of claim 37, wherein values of sets associated with a respective category are numerical values weighted based on genotypes of the sets, and a combination of the numerical values is associated with one or more possible classifications for the respective category.

45. The method of claim 37, wherein:
at least one of the one or more categories is a decision tree category; and
the method further comprises adding or removing a category to or from the one or more categories based on the respective classification assigned to the decision-tree category.

46. The method of claim 37, wherein the respective classification includes one or more of a rating or risk assessment for the corresponding category.

47. The method of claim 37, further comprising:
directing the subject to enact at least one intervention based on the health profile.

48-50. (canceled)

51. The method of claim 37, wherein:
assigning the respective classification includes predicting a health state of the subject based on the values assigned to the plurality of sets, and the health profile information includes the predicted health state; and
the predicted health state includes:
a risk of one or more of irregular ovulation, premature ovarian failure, lifelong risk of estrogen toxicity, polycystic ovarian syndrome, endometriosis, vascular inflammation, fibromyalgia, or breast cancer; or
a risk of one or more of low testosterone, male pattern baldness, benign prostate hyperplasia, ability to develop lean muscle, low libido, or complications from hormone replacement therapy.

52-54. (canceled)
